(19) United States
(12) Patent Application Publication
(10) Pub. No.: US 2013/0208222 A1
(43) Pub. Date: Aug. 15, 2013

(54) REACTIVE MESOGEN COMPOUND, LIQUID CRYSTAL COMPOSITION INCLUDING THE SAME, METHOD OF MANUFACTURING A DISPLAY PANEL, AND DISPLAY PANEL

(71) Applicants: Dae-Ho SONG, Hwaseong-si (KR); Young-Jin PARK, Suwon-si (KR); Gyeong-Eun EOH, Seoul (KR); Sung-Chan JO, Seoul (KR); Sang-Wan SO, Yongin-si (KR)

(72) Inventors: Dae-Ho SONG, Hwaseong-si (KR); Young-Jin PARK, Suwon-si (KR); Gyeong-Eun EOH, Seoul (KR); Sung-Chan JO, Seoul (KR); Sang-Wan SO, Yongin-si (KR)

(73) Assignee: SAMSUNG DISPLAY CO., LTD., Yongin-City (KR)

(21) Appl. No.: 13/587,443

(22) Filed: Aug. 16, 2012

(30) Foreign Application Priority Data
Feb. 13, 2012 (KR) 10-2012-0014351

(51) Int. Cl. C07D 333/24; B29D 11/00; C07D 409/04; G02F 1/1337; C09K 19/34; C07D 333/32

(52) U.S. Cl. 349/123; 252/299.61; 549/79; 549/66; 549/59; 264/1.36

(57) ABSTRACT

A reactive mesogen compound, a liquid crystal composition including the reactive mesogen compound, a method of manufacturing a display panel, and a display panel are disclosed. The reactive mesogen compound may be activated by light having a wavelength between about 300 nm and about 700 nm.

### PRIORITY STATEMENT

This application claims priority to Korean Patent Application No. 10-2012-0014351, filed on Feb. 13, 2012 and all the benefits accruing therefrom under 35 U.S.C. §119, the contents of which in their entirety are herein incorporated by reference.

### BACKGROUND OF THE INVENTION

1. Field of the Invention

Disclosed herein is a reactive mesogen compound, a liquid crystal composition including the reactive mesogen compound, a method of manufacturing a display panel, and a display panel. More particularly, this disclosure relates to a reactive mesogen compound reacted with a light, a liquid crystal composition including the reactive mesogen compound, a method of manufacturing a display panel, and a display panel.

2. Description of the Related Art

Generally, a liquid crystal display (“LCD”) panel includes a first display substrate including a switching element for driving each of the pixels, a second display substrate facing the first display substrate, and a liquid crystal (“LC”) layer interposed between the first and second display substrates. A voltage is applied to the LC layer to control light transmittance so that the LCD panel displays an image.

A super vertical alignment (“SVA”) mode is a technology that controls LC molecules pretilted using a reactive mesogen compound between the first and second display substrates. For example, the LC molecules and the reactive mesogen compound are interposed between the first and second display substrates, and a light is provided to the first and second display substrates whilst applying voltages to the first and second display substrates, so that the LC molecules are pretilted. In other words, the reactive mesogen compound is polymerized (hardened) by the light of an ultraviolet (“UV”) ray to be pretilted.

However, when the reactive mesogen compound is irradiated with UV rays in order to polymerize it, the light has to pass through base glass substrates of the first and second display substrates to reach the reactive mesogen compound. The glass substrate may absorb and/or reflect the UV rays, thereby reducing the photo-reactive efficiency of the reactive mesogen compound. The main wavelengths absorbed by the reactive mesogen compound are greater than or equal to about 200 nm and less than about 300 nm. As a result, to the time utilized to polymerize the reactive mesogen compound needs to be increased in order to compensate for the light absorbed and/or reflected by the glass. This reduces productivity in the manufacturing process for producing the display apparatus.

### SUMMARY OF THE INVENTION

Disclosed herein is a reactive mesogen compound which has a high absorption for an UV ray of a long wavelength range.

Disclosed herein too is a liquid crystal composition which includes the reactive mesogen compound.

Disclosed herein too is a method of manufacturing a display panel using the reactive mesogen compound.

According to one aspect of the present invention, a reactive mesogen compound is represented by Chemical Formula 1-1.

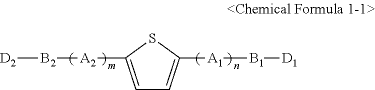

In Chemical Formula 1-1, each of A₁ and A₂ independently represents

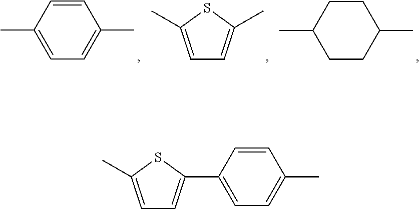

or a single bond, each of “n” and “m” independently represents an integer in a range of 0 to about 3 (“m” and “n” do not simultaneously represent 0), each of B₁ and B₂ independently represents a single bond or —(CH₂)ₖ— (“k” represents an integer in a range of 0 to 6), at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

In an embodiment, the reactive mesogen compound may be activated by light having a wavelength between about 300 nm and about 700 nm.

According to anther aspect of the present invention, a liquid crystal composition includes liquid crystal molecules including a compound having at least one carbon ring and a reactive mesogen compound represented by Chemical Formula 1-1.

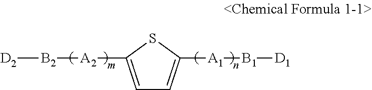

In Chemical Formula 1-1, each of A₁ and A₂ independently represents

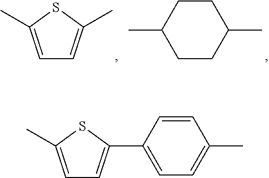

or a single bond, each of “n” and “m” independently represents an integer in a range of 0 to about 3 (“m” and “n” do not simultaneously represent 0), each of B₁ and B₂ independently represents a single bond or —(CH₂)ₖ— (“k” represents an integer in a range of 0 to 6), at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

In an embodiment, an amount of the liquid crystal molecules may be about 95% by weight to about 99.9% by weight based on a total weight of the liquid crystal composition, and an amount of the reactive mesogen compound may be about 0.1% by weight to about 5% by weight based on a total weight of the liquid crystal composition.

In an embodiment, the reactive mesogen compound may be activated by a light having a wavelength between about 300 nm and about 700 nm.

According to still another aspect of the present invention, a method of manufacturing a display panel is provided. In the method, a first display substrate is formed, which includes a pixel electrode and a first alignment layer disposed on the pixel electrode. A second display substrate is formed, which faces the first display substrate and includes a second alignment layer. A liquid crystal composition is interposed between the first and second display substrates, and the liquid crystal composition includes liquid crystal molecules having at least one carbon ring and a reactive mesogen compound represented by Chemical Formula 1-1. The liquid crystal composition is irradiated with light and the liquid crystal molecules are pretilted by an electric field between the first and second display substrates.

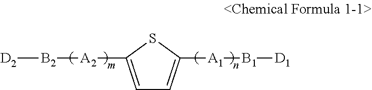

In Chemical Formula 1-1, each of A₁ and A₂ independently represents

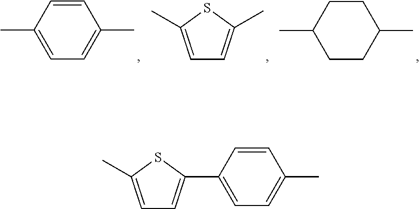

or a single bond, each of “n” and “m” independently represents an integer in a range of 0 to about 3; “m” and “n” do not simultaneously represent 0, each of B₁ and B₂ independently represents a single bond or —(CH₂)ₖ—; wherein “k” represents an integer in a range of 0 to about 6, at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

In an embodiment, the light may be irradiated on to the liquid crystal composition in a non-electric state between the first and second display substrates after first being irradiated on to the liquid crystal composition.

In an embodiment, in irradiating the liquid crystal composition, the reactive mesogen compound may be reacted by the irradiant light to form a first mesogen polymer layer on the first alignment layer, and the first mesogen polymer layer may fix the liquid crystal molecules disposed adjacent to the first display substrate so that the liquid crystal molecules have a pretilted angle on a surface of the first mesogen polymer layer. Simultaneously, the reactive mesogen compound may be reacted by the irradiant light to form a second mesogen polymer layer on the second alignment layer, and the second mesogen polymer layer may fix the liquid crystal molecules disposed adjacent to the second display substrate so that the liquid crystal molecules have a pretilted angle on a surface of the second mesogen polymer layer.

According to still another aspect of the present invention, a method of manufacturing a display panel is provided. In the method, a first alignment layer is disposed on a pixel electrode of a first display substrate using a composition for manufacturing an alignment layer, where the composition includes an alignment polymer having a functional group represented by Chemical Formula 1-2. A second alignment layer of a second display substrate facing the first display substrate is formed using the composition. A liquid crystal composition is interposed between the first and second display substrates. A light is irradiated on to the first and second display substrates, between which an electric field is formed, to cause a photo-reaction of the functional group of the first and second alignment layers.

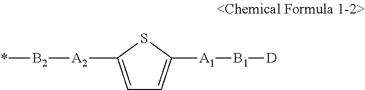

In Chemical Formula 1-2, each of each of A₁ and A₂ independently represents

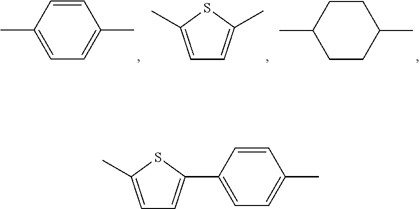

or a single bond, each of “n” and “m” independently represents an integer in a range of 0 to about 3 “m” and “n” do not simultaneously represent 0, each of B₁ and B₂ independently represents a single bond or —(CH₂)ₖ—; wherein “k” represents an integer in a range of 0 to 6, at least one —(CH₂)— in each of B₁ and B₂ is replaceable with —O—, —COO—, —OCO— or —CO—, D independently represents methacrylate or acrylate, and at least one hydrogen atom of each of A₁, A₂ and 2,5-thiophene is replaceable with F, Cl, OCH₃ or an alkyl group having 1 to about 6 carbon atoms.

In an embodiment, as a result of the irradiation with light, a first mesogen polymer layer may be disposed on the first alignment layer, and the first mesogen polymer layer may fix the liquid crystal molecules disposed adjacent to the first display substrate so that the liquid crystal molecules have a pretilted angle on a surface of the first mesogen polymer layer. Simultaneously, a second mesogen polymer layer may be disposed on the second alignment layer, the second mesogen polymer fixing the liquid crystal molecules disposed adjacent to the second display substrate so that the liquid crystal molecules have a pretilted angle on a surface of the second mesogen polymer layer.

In an embodiment, the second alignment layer may be formed using the composition that is used for forming the alignment layer.

According to the present invention, although using a glass substrate as a base substrate, a photo-reactive efficiency of a reactive mesogen compound may be improved in a process providing a light to the reactive mesogen compound, for the light having greater wavelength than a wavelength absorbed in the glass substrate. That is, the reactive mesogen compound is activated by a light having a wavelength in a range of about 300 nm to about 700 nm, and an intensity of the reactive mesogen compound for the light having a wavelength between about 300 nm and about 370 nm may be maximized. Thus, a polymerization time of the reactive mesogen compound is decreased so that an entire manufacturing time may be decreased to improve the productivity. In addition, the reactive mesogen compound is almost reacted with a light so that an amount of the reactive mesogen compound remained in an LC layer may be minimized to improve reliability of a manufacturing process.

### BRIEF DESCRIPTION OF THE DRAWINGS

The above and other features and advantages of the present invention will become more apparent by describing in detailed example embodiments thereof with reference to the accompanying drawings, in which:

FIG. 1 is a nuclear magnetic resonance (“NMR”) graph of a compound manufactured from a reactive mesogen compound according to Example 1 of the present invention;

FIG. 2 is a mass spectrometry (“MS”) graph of the compound manufactured from the reactive mesogen compound according to Example 1 of the present invention;

FIG. 3 is a graph representing a absorption ratio along a wavelength according to Example 1 and Example 2 of the present invention;

FIG. 4 is a plan view illustrating a display panel manufactured according to an exemplary embodiment of the present invention;

FIG. 5 is a cross-sectional view taken along a line I-I′ in FIG. 4;

FIG. 6 and FIG. 7 are conceptual views illustrating a method of manufacturing the display panel in FIG. 5;

FIG. 8 is a flow chart illustrating another method of manufacturing the display panel in FIG. 5;

FIG. 9 is a cross-sectional view illustrating a display panel manufactured according to another exemplary embodiment of the present invention;

FIG. 10 is a plane view illustrating a display panel manufactured according to another exemplary embodiment of the present invention; and

FIG. 11 is a cross-sectional view taken along a line II-II′ in FIG. 10.

### DETAILED DESCRIPTION OF THE INVENTION

### Reactive Mesogen Compound

A reactive mesogen compound is a compound including thiophene and is represented by Chemical Formula 1-1.

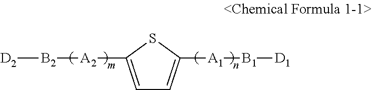

In Chemical Formula 1-1, each of A₁ and A₂ independently represents

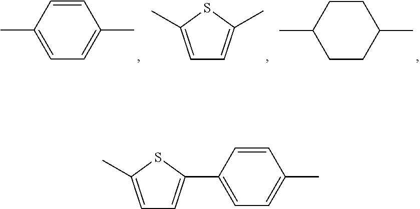

or a single bond, each of “n” and “m” independently represents an integer in a range of 0 to about 3. Here, “m” and “n” do not simultaneously represent 0. In Chemical Formula 1-1, A₁, A₂ and 2,5-thiophene may determine a backbone of the reactive mesogen compound.

Each of B₁ and B₂ independently represents a single bond or —(CH₂)ₖ—. Here, “k” represents an integer in a range of 0 to about 6. At least one —(CH₂)— in each of B₁ and B₂ is replaceable with —O—, —COO—, —OCO— or —CO—.

In Chemical Formula 1-1, each of D₁ and D₂ is a photo-functional group that is substantially activated by a light in the reactive mesogen compound. The photo-functional group is activated by the light to polymerize the reactive mesogen compound. Each of D₁ and D₂ independently includes an ethylenically unsaturated moiety (e.g., a methacrylate or an acrylate). Each of D₁ and D₂ may be connected to the backbone of the reactive mesogen compound by each of B₁ and B₂. That is, each of B₁ and B₂ may function as a spacer in the reactive mesogen compound.

In Chemical Formula 1-1, at least one hydrogen atom of each of A₁, A₂ and 2,5-thiophene is replaceable with F, Cl, OCH₃ or an alkyl group having 1 to about 6 carbon atoms.

Since the reactive mesogen compound includes thiophene having sulfur (S) which has an unshared electron pair, the conjugational effect (i.e., the tendency to conjugate) of the thiophene exceeds that of a phenyl group because of the presence of the unshared electron pair of sulfur. When the conjugation effect is increased, an energy gap of the reactive mesogen compound is decreased. This reduction in the energy gap permits an increase in the wavelength of a light absorbed in the reactive mesogen compound. That is, the wavelength of the light absorbed by the reactive mesogen compound may be increased to be greater than or equal to about 300 nm. The reactive mesogen compound includes thiophene to be activated by the light having a wavelength in a range of about 300 nm to about 700 nm and to have a maximal absorption between about 300 nm and about 370 nm.

Since the reactive mesogen compound includes thiophene, the conjugation effect of the reactive mesogen compound is increased and the wavelength absorbed by the reactive mesogen compound is increased. When the reactive mesogen compound is disposed on a surface of a glass surface as a base substrate and a light is provided to a different surface from the surface, a photo-reactive efficiency of the reactive mesogen compound may be improved although the glass substrate partially absorbs the light, since the wavelength range in which the reactive mesogen compound is activated by the light is different from a wavelength range absorbed by the glass substrate.

Examples of the reactive mesogen compound may include compounds represented by Chemical Formula 2-1, Chemical Formula 2-2 and Chemical Formula 2-3, when each of A₁ and A₂ independently represents

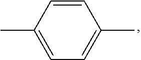

and each of “m” and “n” independently represents 1 in Chemical Formula 1-1.

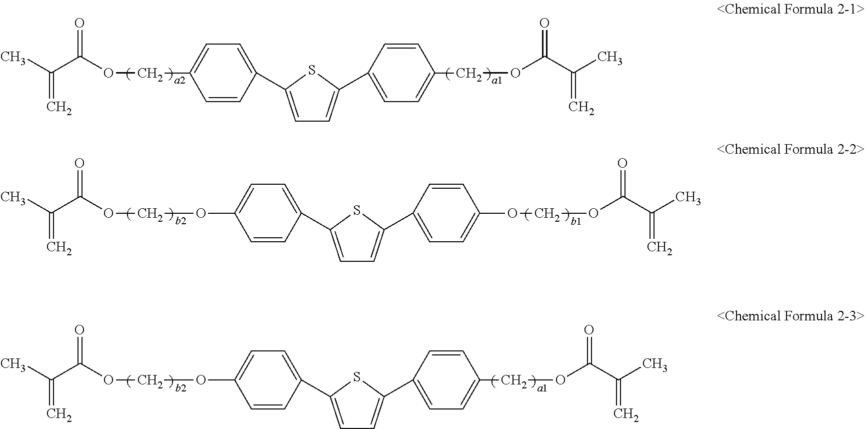

In each of Chemical Formula 2-1 to Chemical Formula 2-3, each of a1 and a2 independently represents an integer in a range of 0 to about 6, and each of b1 and b2 independently represents an integer in a range of 1 to about 5. Although B₁ and B₂ as the spacer of Chemical Formula 1-1 include “—O—”, the “—O—” is connected to a bond “—O—” of methacrylate as the photo-reactive group to be “—O—O—” when end portions of B₁ and B₂ connected to D₁ and D₂ as the photo-reactive group includes the structure of “—O—”, respectively. When the reactive mesogen compound includes a structure sequentially connecting two oxygen atoms to each other, the stability of the reactive mesogen compound may be decreased by the presence of a structure comprising “—O—O—.” Thus, B₁ and B₂ as the spacer of Chemical Formula 1-1 include the structure of “—O—,” however the end portions of B₁ and B₂ connected to D₁ and D₂ preferably include at least one —CH₂—. Therefore, each of b1 and b2 in each of Chemical Formula 2-1 to Chemical Formula 2-3 does not represent 0 and merely represents an integer in a range of 1 to about 5.

The photo-reactive group represents methacrylate in Chemical Formula 2-1 to Chemical Formula 2-3. Alternatively, the photo-reactive group may represent acrylate. In other words, the photoreactive group contains an ethylenic unsaturation that may facilitate the polymerization of the composition when irradiated by the appropriate source (wavelength) of light.

Examples of the reactive mesogen compound may include compounds represented by Chemical Formula 3-1, Chemical Formula 3-2, Chemical Formula 3-3 and Chemical Formula 3-4 when A₁ represents

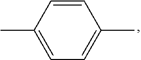

A₂ represents

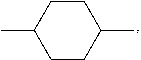

and each of “m” and “n” represents 1 in Chemical Formula 1-1.

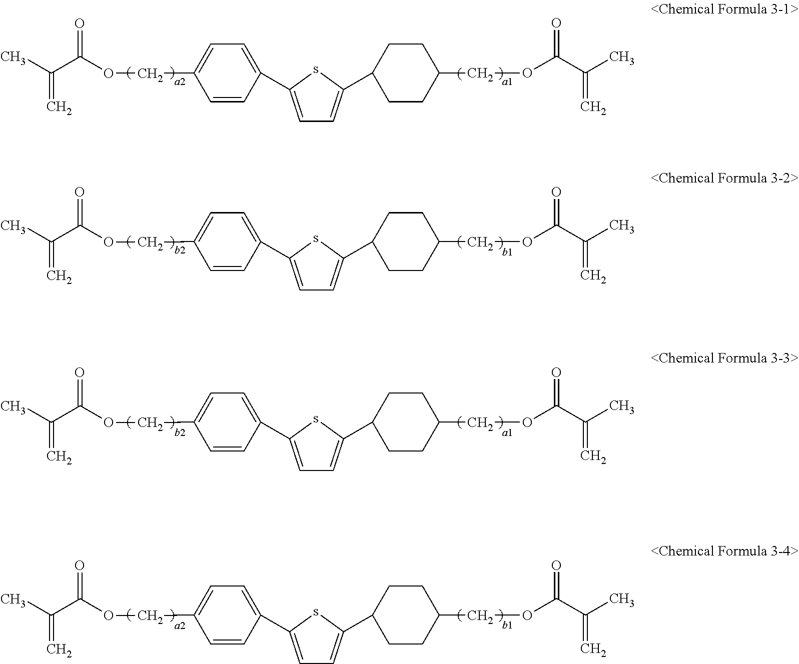

In each of Chemical Formula 3-1 to Chemical Formula 3-4, each of a1 and a2 independently represents an integer in a range of 0 to about 6, and each of b1 and b2 independently represents an integer in a range of 1 to about 5. In Chemical Formula 3-1 to Chemical Formula 3-4, b1 and b2 do not represent 0 for substantially the same reason as illustrated above in Chemical Formula 2-1 to Chemical Formula 2-3. Thus, each of b1 and b2 independently represents an integer in a range of 1 to 5. The photo-reactive group represents methacrylate in Chemical Formula 3-1 to Chemical Formula 3-4. Alternatively, the photo-reactive group may represent acrylate.

Examples of the reactive mesogen compound may include compounds represented by Chemical Formula 4-1, Chemical Formula 4-2, Chemical Formula 4-3 and Chemical Formula 4-4 when A₁ represents

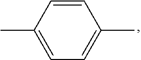

A₂ represents a single bond, “m” represents 2 and “n” represents 1 in Chemical Formula 1-1.

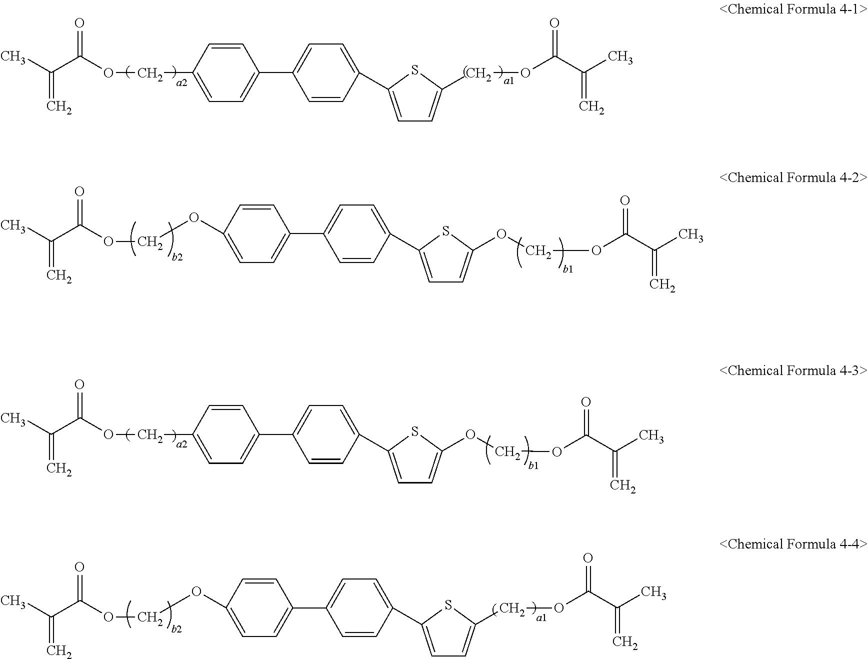

In Chemical Formula 4-1 to Chemical Formula 4-4, each of a1 and a2 independently represents an integer in a range of 0 to about 6, and each of b1 and b2 independently represents an integer in a range of 1 to about 5. The photo-reactive group represents methacrylate in Chemical Formula 4-1 to Chemical Formula 4-4. Alternatively, the photo-reactive group may represent acrylate.

Examples of the reactive mesogen compound may include compounds represented by Chemical Formula 5-1, Chemical Formula 5-2, Chemical Formula 5-3 and Chemical Formula 5-4 when A₁ represents

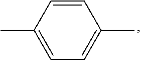

A₂ represents a single bond, each of “m” and “n” independently represents 1 in Chemical Formula 1-1.

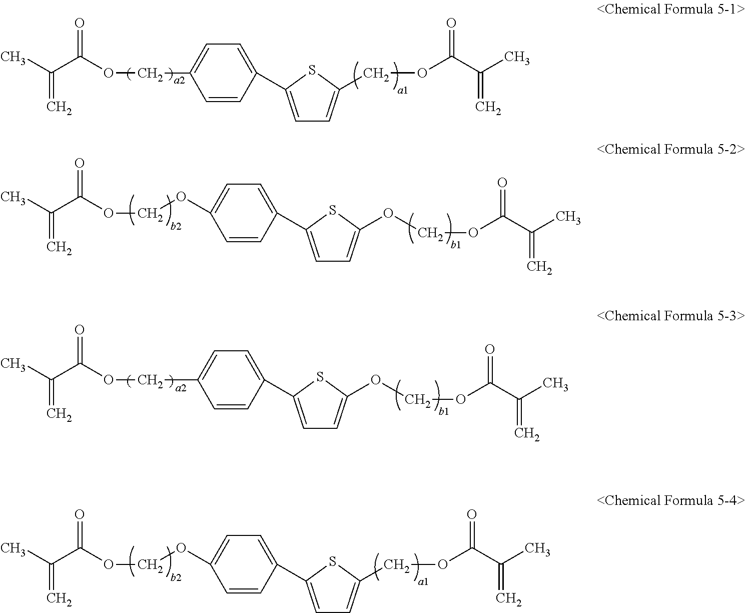

In Chemical Formula 5-1 to Chemical Formula 5-4, each of a1 and a2 independently represents an integer in a range of 0 to about 6, and each of b1 and b2 independently represents an integer in a range of 1 to about 5.

Examples of the reactive mesogen compound may include compounds represented by Chemical Formula 6-1, Chemical Formula 6-2, Chemical Formula 6-3 and Chemical Formula 6-4 when A₁ represents

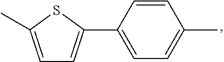

A₂ represents

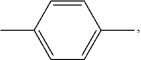

and each of “m” and “n” independently represents 1 in Chemical Formula 1-1.

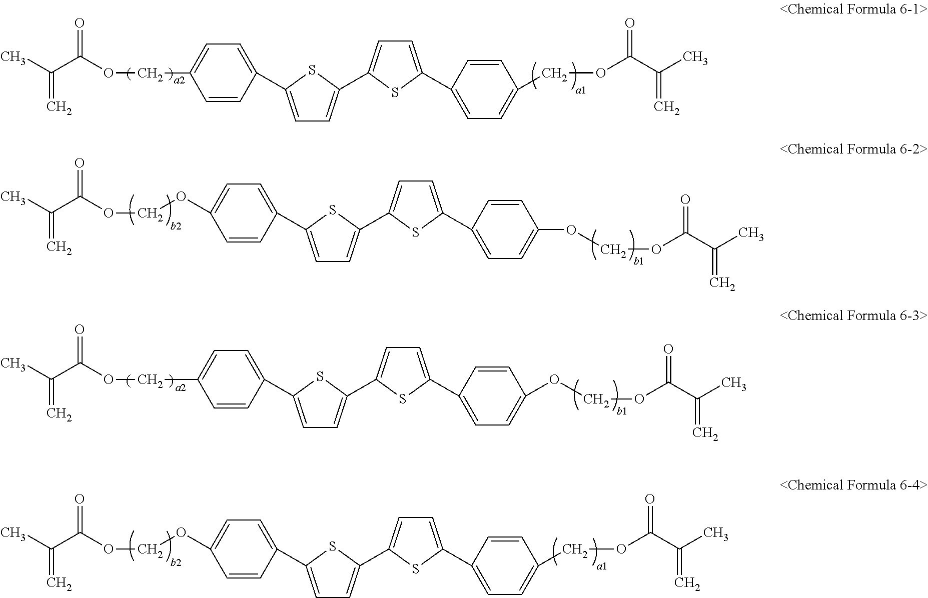

Each of Chemical Formula 6-1 to Chemical Formula 6-4, each of a1 and a2 independently represents an integer in a range of 0 to about 6, and each of b1 and b2 independently represents an integer in a range of 1 to about 5.

According to the above descriptions, a backbone of a reactive mesogen compound includes thiophene so that the wavelength of light for activating the reactive mesogen compound may be increased, compared to a reactive mesogen compound that includes a phenyl group and/or a cyclohexyl group. The reactive mesogen compound may be activated by a light in a range of about 300 nm to about 700 nm, and an intensity of the reactive mesogen compound for a light having a wavelength between about 300 nm and about 370 nm may therefore be maximized.

Hereinafter, a photo-reactivity of the reactive mesogen compound will be illustrated with a synthetic example of the reactive mesogen compound according to the present invention.

### Manufacturing a Reactive Mesogen Compound

A reactive mesogen compound including thiophene and represented by Chemical Formula 2-4 according to Example 1 of the present invention was prepared using about 55 g of dibromothiophene having a molecular weight of about 241.93 grams per mole and about 50 g of 4-methylphenylboronic acid hydrate having a molecular weight of about 151.96 grams per mole as Reaction Formula 1. An amount of the reactive mesogen compound represented by Chemical Formula 2-4 manufactured by Reaction Formula 1 was about 10 g.

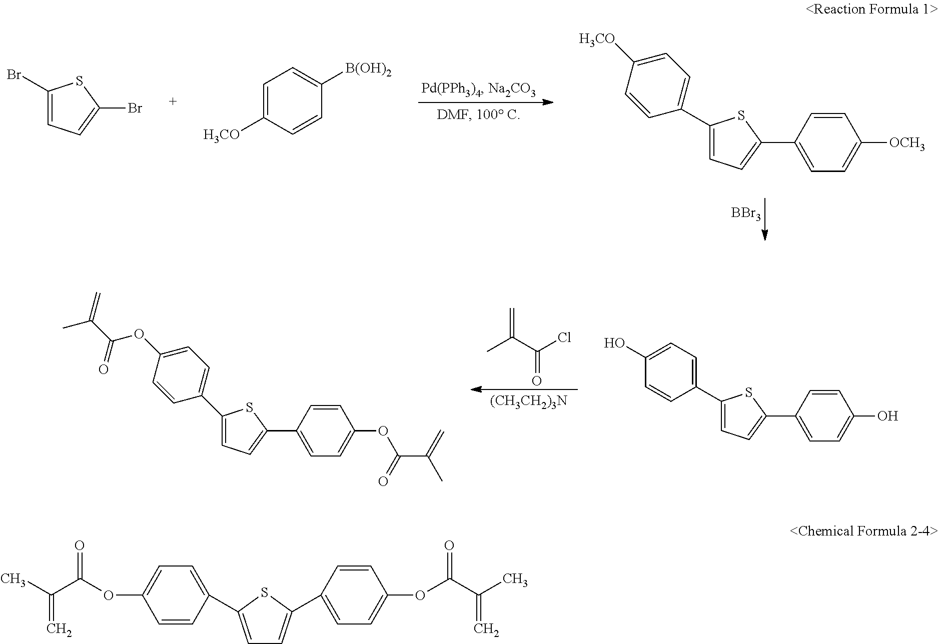

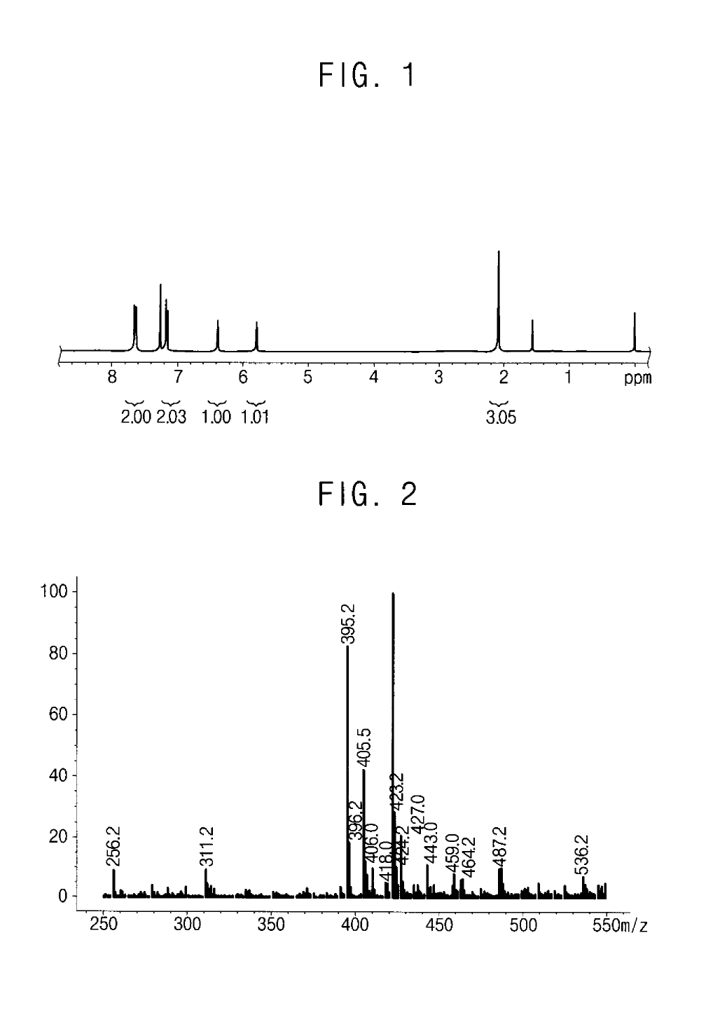

In Reaction Formula 1, “Pd(PPh₃)₄” represents Tetrakis(triphenylphosphine) palladium, and “DMF” represents dimethylformamide. After a product synthesized according to Reaction Formula 1 was dispersed in chloroform-D (CDCl₃) as an assay solvent, a structure of the product was analyzed using a nuclear magnetic resonance (“NMR”) apparatus and a mass spectrometry (“MS”) was performed. Obtained results are illustrated in FIG. 1 and FIG. 2.

### Analysis Results of NMR

FIG. 1 is a nuclear magnetic resonance (“NMR”) graph of a compound manufactured by a synthetic example of a reactive mesogen compound according to Example 1 of the present invention. In FIG. 1, an x axis represents a delta (δ, unit: ppm) and a y axis represents absorption intensity.

Referring to FIG. 1, peaks in a value of a delta (δ) of about 7.63 ppm and about 7.15 ppm show that the product includes a hydrogen atom of a phenyl ring, and a peak in a value of the delta in about 7.25 ppm shows that the product includes a hydrogen atom of a thiophene ring and the assay solvent. In addition, peaks in a value of a delta (δ) of about 6.37 ppm and about 5.78 ppm show that the product includes a hydrogen atom connected to a carbon atom of a double bond in methacrylate, and a peak in a value of a delta (δ) of about 2.08 ppm shows that the product includes a hydrogen atom of a methyl group connected to a carbon atom of a double bond in methacrylate.

### Analysis Results of MS

FIG. 2 is a mass spectrometry (“MS”) graph of the compound manufactured by the synthetic example of the reactive mesogen compound according to Example 1 of the present invention.

In FIG. 2, an “x” axis represent “m/z” as a ratio of a mass for a charge, and a “y” axis represents an abundance of a decomposed material. Referring to FIG. 2, when the “m/z” is adjacent to about 395.2, about 405 and about 422.2, a value of the y axis is greater than the other range.

From the graphs of FIG. 1 and FIG. 2, the reactive mesogen compound represented by Chemical Formula 2-4 is actually manufactured via a process shown in Reaction Formula 1. In particular, a peak in the “m/z” of about 405 shows a compound represented by Chemical Formula 2-4, and peaks in the “m/z” in a range of about 422.2 to about 427 shows that the product includes a compound (M-Na) combining the compound represented by Chemical Formula 2-4 with sodium (Na). Therefore, the reactive mesogen compound according to Example 1 of the present invention represented by Chemical Formula 2-4 and having a theoretical molecular weight of about 404.4 may be manufactured by the above results.

### Measurement of a Light Absorption Ratio

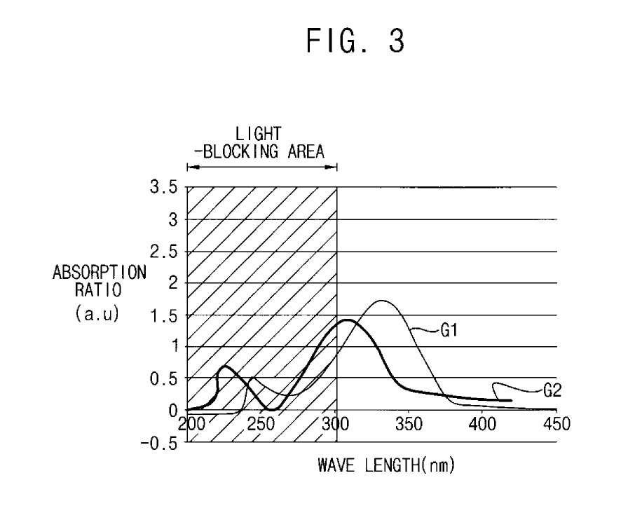

A light absorption ratio of the reactive mesogen compound represented by Chemical Formula 2-4 according to Example 1 of the present invention was measured along a wavelength, and thus obtained results are illustrated in FIG. 3.

FIG. 3 is a graph representing an absorption ratio along a wavelength according to Example 1 and Example 2 of the present invention.

In FIG. 3, “G1” is a graph for the reactive mesogen compound represented by Chemical Formula 2-4 according to Example 1 of the present invention. In FIG. 3, “G2” is a simulation graph for a reactive mesogen compound represented by Chemical Formula 4-5 according to Example 2 of the present invention.

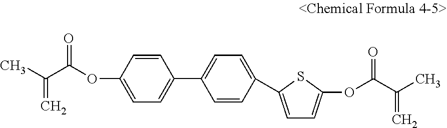 [SMILES: C=C(C)C(=O)Oc1ccc(-c2ccc(-c3ccc(OC(=O)C(=C)C)s3)cc2)cc1]

Referring to FIG. 3, a reactive mesogen compound represented by Chemical Formula 2-4 has a maximal absorption in a wavelength between about 320 nm and about 350 nm. In addition, a reactive mesogen compound represented by Chemical Formula 4-5 has a maximal absorption in a wavelength between about 300 nm and about 320 nm. The experimental and simulation results show that a wavelength of a maximal absorption ratio of the reactive mesogen compound is increased, compared to a reactive mesogen compound including a bond of phenyl group-phenyl group as a main ring structure and having a wavelength in a maximal absorption ratio of less than about 300 nm, particularly in a range of about 260 nm to about 280 nm.

According to the above descriptions, the reactive mesogen compound of the present invention may be activated by a light having a wavelength in a range of about 300 nm to about 700 nm and a light intensity of a wavelength between about 300 nm and about 370 nm may be increased. Thus, a polymerization time of the reactive mesogen compound, that is, a photo-reaction time of the reactive mesogen compound is decreased so that the entire manufacturing time is decreased to improve the productivity. In addition, the reactive mesogen compound may be reacted with the light to minimize an amount of the reactive mesogen compound remained in an LC layer, thereby improving a reliability of a manufacturing process.

### LC Composition

An LC composition according to the present invention includes LC molecules and a reactive mesogen compound represented by Chemical Formula 1-1.

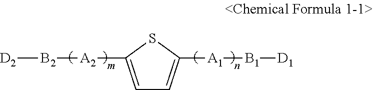

In Chemical Formula 1-1, each of A₁ and A₂ independently represents

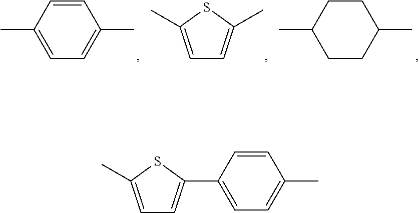

or a single bond, each of “n” and “m” independently represents an integer in a range of 0 to about 3 (“m” and “n” do not simultaneously represent 0), each of B₁ and B₂ independently represents a single bond or —(CH₂)ₖ— (“k” represents an integer in a range of 0 to 6), at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

The reactive mesogen compound represented by Chemical Formula 1-1 used in the LC composition is substantially the same as the reactive mesogen compound illustrated above. Thus, any repetitive descriptions will be omitted.

The LC molecules include a compound having at least one carbon ring. Examples of the carbon ring may include phenyl group, cyclohexyl group, and the like. Each of the LC molecules may include a structure of “phenyl-phenyl,” “phenyl-cyclohexyl” or “cyclohexyl-cyclohexyl.” A kind of the LC molecules should not be construed as limited to the examples set forth herein, and the LC molecules may use a known LC composition for an LCD panel.

When an amount of the reactive mesogen compound is less than about 0.1% by weight based on a total weight of the LC composition, the LC molecules may be not stably disposed having a pretilted angle by the reactive mesogen compound in a process forming the LC layer using the LC composition. In addition, when the amount of the reactive mesogen compound is greater than about 5% by weight based on the total weight of the LC composition, the reactive mesogen compound may affect the LC molecules so that the LC molecules is difficult to be controlled. Thus, the amount of the reactive mesogen compound may be preferably in a range of about 0.1% by weight to about 5% by weight.

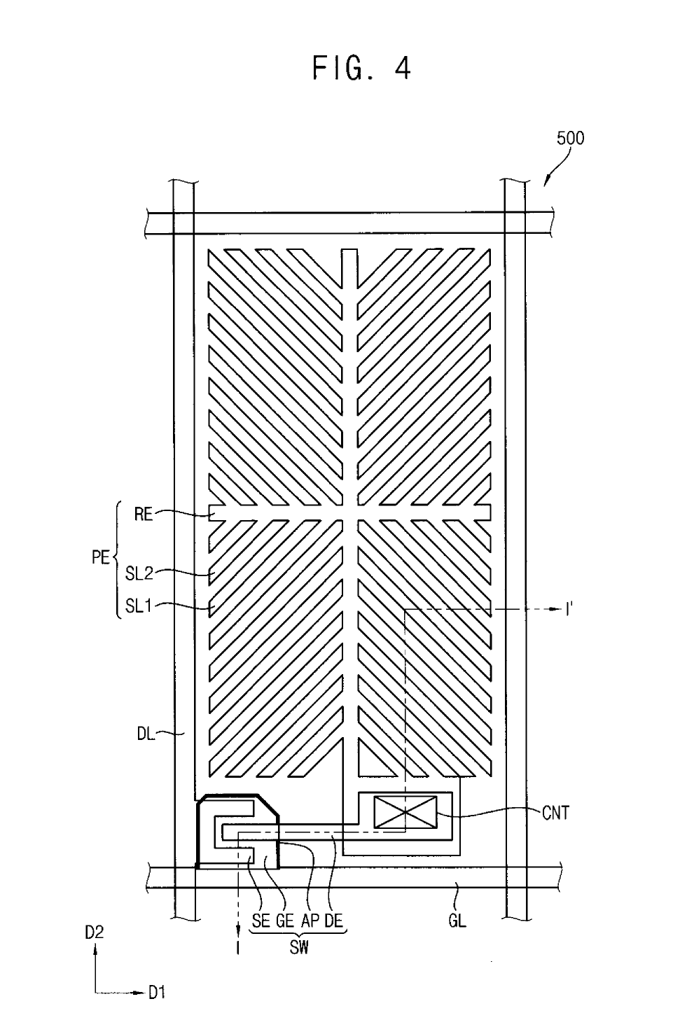

A method of manufacturing a display panel using the LC composition will be illustrated later referring to FIG. 4 to FIG. 7.

### Composition for Forming an Alignment Layer

A composition for forming an alignment layer according to the present invention includes an alignment polymer including a functional group represented by Chemical Formula 1-2 as a side chain.

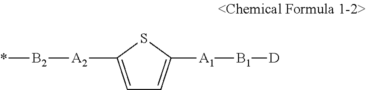

In Chemical Formula 1-2, each of each of A₁ and A₂ independently represents

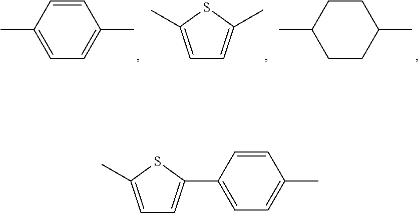

or a single bond, each of “n” and “m” independently represents an integer in a range of 0 to 3 (“m” and “n” do not simultaneously represent 0), each of B₁ and B₂ independently represents a single bond or —(CH₂)ₖ— (“k” represents an integer in a range of 0 to 6), at least one —(CH₂)— in each of B₁ and B₂ is replaceable with —O—, —COO—, —OCO— or —CO—, D independently represents methacrylate or acrylate, and at least one hydrogen atom of each of A₁, A₂ and 2,5-thiophene is replaceable with F, Cl, OCH₃ or an alkyl group having 1 to about 6 carbon atoms.

In Chemical Formula 1-2, “D” is a photo-reactive group substantially activated by a light. The photo-reactive group is activated to polymerize the reactive mesogen compound. “D” is connected to a backbone of the functional group via B₁.

When the functional group includes a structure sequentially connecting oxygen atoms such as —O—O—, the photo-reactivity may be decreased so that an end portion of “D” as the photo-reactive group or an end portion of the functional group connected to the main chain preferably include at least a bond —CH₂— although B₁ or B₂ includes a structure —O—.

In a functional group represented by Chemical Formula 1-2, a photo-characteristic of the functional group is substantially the same with that of the reactive mesogen compound of the present invention, except for connecting “B₂” of the Chemical Formula 1-2 to a polymer as a main chain. In particular, an alignment polymer including the functional group represented by Chemical Formula 1-2 may be activated by a light having a wavelength in a range of about 300 nm to about 700 nm and may have a maximal absorption for light between about 300 nm and about 370 nm, similar to the reactive mesogen compound represented by Chemical Formula 1-1 of the present invention. Thus, any repetitive descriptions for the photo-characteristic will be omitted.

In the alignment polymer, examples of the main chain may include polyimide, polyamic acid, polysiloxane, polyvinylcinnamate, polyacrylate, polymethylmethacrylate, or the like. However, a kind of the main chain should not be construed as limited to the examples set forth herein. The alignment polymer includes the polymers illustrated above as the main chain, the functional group represented by Chemical Formula 1-2 as the side chain is connected to the main chain.

The composition for forming the alignment layer further includes a solvent for dispersing the alignment polymer. For example, an amount of the alignment polymer may be less than or equal to about 30% by weight based on a total weight of the composition for forming the alignment layer, with the solvent as the remainder of the composition.

A method of manufacturing a display panel using the composition for forming the alignment layer will be illustrated later referring to FIG. 8.

### Method of Manufacturing a Display Panel

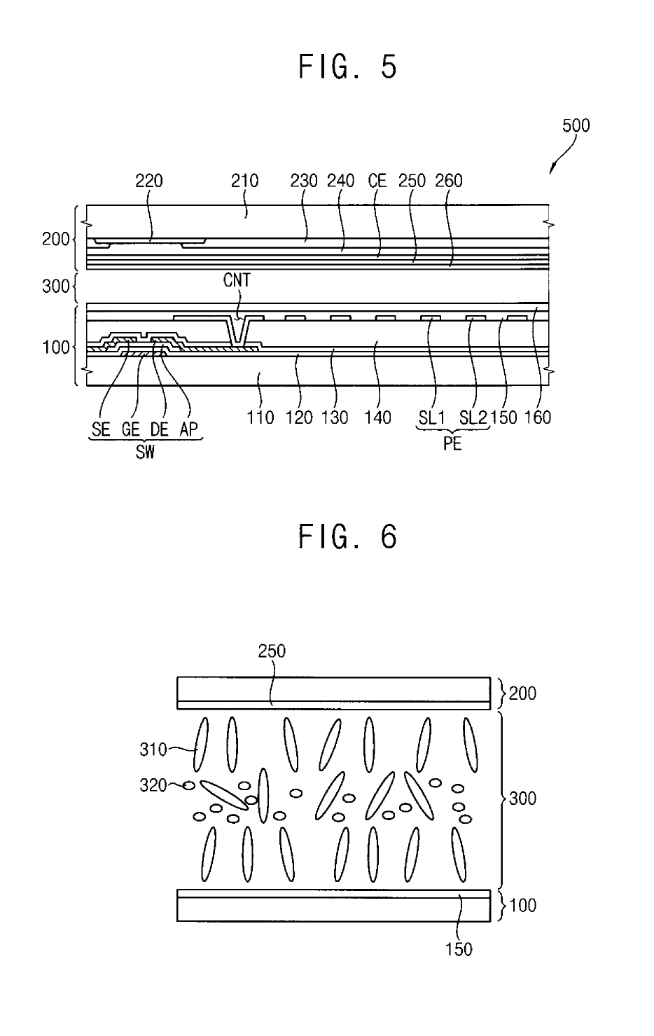

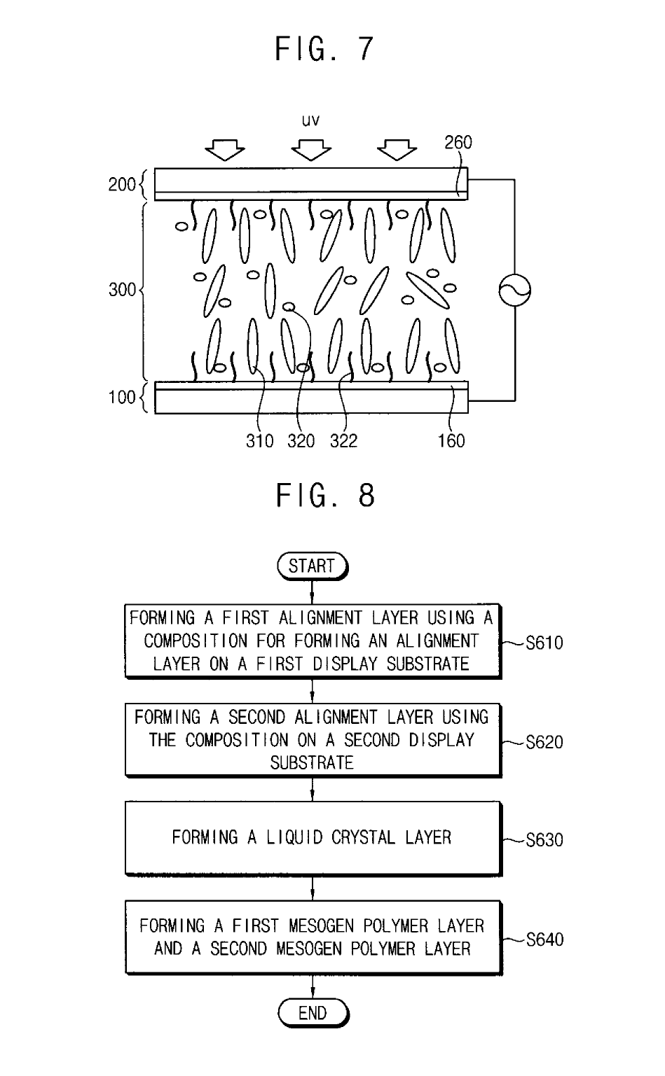

Hereinafter, a method of manufacturing a display panel will be explained in detail with reference to the accompanying drawings. After a structure of a display panel manufactured according to the present invention is shortly explained referring to FIG. 4 and FIG. 5, a first method of manufacturing the display panel will be explained referring to FIG. 6 and FIG. 7 and a second method will be explained referring to FIG. 8.

FIG. 4 is a plan view illustrating a display panel manufactured according to an example embodiment of the present invention.

FIG. 5 is a cross-sectional view taken along a line I-I′ in FIG. 4.

Referring to FIG. 4 and FIG. 5, a display panel 500 includes a first display substrate 100, a second display substrate 200 and an LC layer 300.

The first display substrate 100 includes a gate line GL disposed on a first base substrate 110, a first insulating layer 120, a data line DL, a switching element SW, a second insulating layer 130, an organic layer 140, a pixel electrode PE, a first alignment layer 150 and a first mesogen polymer layer 160.

The gate line GL extends along a first direction D1 on the first base substrate 110. The data line DL extends along a second direction D2 crossing the gate line GL. The first insulating layer 120 is disposed on the gate line GL and insulates the gate line GL with the data line DL.

The switching element SW includes a gate electrode GE connected to the gate line GL, an active pattern AP disposed on the first insulating layer 120 corresponding to the gate electrode GE, a source electrode SE connected to the data line DL, and a drain electrode DE contacting with the pixel electrode PE. The source electrode SE and the drain electrode DE are spaced apart from each other. The drain electrode DE contacts with the pixel electrode PE through a contact hole CNT passing through the second insulating layer 130 and the organic layer 140.

The second insulating layer 130 covers the data line DL and the switching element SW. The organic layer 140 is formed between the second insulating layer 130 and the pixel electrode PE.

The pixel electrode PE is disposed on the organic layer 140. The pixel electrode PE may include transparent and conductive material. The pixel electrode PE contacts with the drain electrode DE through the contact hole CNT. Thus, the switching element SW may be connected to the pixel electrode PE. The pixel electrode PE includes a connecting electrode portion RE and fine-electrode portions SL1 and SL2.

The connecting electrode portion RE is connected to the drain electrode DE of the switching element SW through the contact hole CNT. The connecting electrode portion RE divides a pixel to a plurality of sub regions. For example, the connecting electrode portion RE may have the cross shape. The pixel may be divided to four sub regions by the connecting electrode portion RE.

The fine-electrode portions SL1 and SL2 may extend from the connecting electrode portion RE toward an outline of the pixel. The fine-electrode portions SL1 and SL2 are connected to each other by the connecting electrode portion RE. For example, a width of each of the fine-electrode portions SL1 and SL2 may be about 0.1 μm to about 10 μm. A first fine-electrode SL1 and a second fine-electrode SL2 adjacent to each other of the fine-electrode portions SL1 and SL2 may define a “slit” controlling an alignment direction of the LC molecules of the LC layer 300. When the pixel is divided to four sub regions by the connecting electrode portion RE, the fine-electrode portions SL1 and SL2 may respectively extend to directions slanted by about 45°, about 135°, about 225° and about 315° with respect to the connecting electrode portion RE extending in a parallel direction with the gate line GL.

The first alignment layer 150 is disposed on the pixel electrode PE. The first alignment layer 150 may be formed by coating a composition for forming an alignment layer on the first base substrate 110 on which the pixel electrode PE is formed.

The first mesogen polymer layer 160 is disposed on the first alignment layer 150. The first mesogen polymer layer 160 is formed between the first alignment layer 150 and the LC layer 300. The first mesogen polymer layer 160 is disposed on the first alignment layer 150 to fix the LC molecules of the LC layer 300 adjacent to the first display substrate 100 so that the LC molecules of the LC layer 300 have a pretilted angle on the first mesogen polymer layer 160.

The second display substrate 200 faces the first display substrate 100 and interposes the LC molecules with the first display substrate 100. The second display substrate 200 includes a second alignment layer 250 disposed on a second base substrate 210 and a second mesogen polymer layer 260. The second display substrate 200 may further include a light-blocking pattern 220, a color filter 230, an over-coating layer 240 and a common electrode CE which are formed under the second alignment layer 250.

The light-blocking pattern 220 is disposed on the second base substrate 210 corresponding to the gate line GL, the data line DL and the switching element SW of the first display substrate 100. The color filter 230 is disposed on the second base substrate 210 corresponding to a region in which the pixel electrode PE is formed. The over-coating layer 240 is formed between the common electrode CE and the second base substrate 210 on which the light-blocking pattern 220 and the color filter 230 are formed. The common electrode CE includes a transparent conductive material and is entirely disposed on the second base substrate 210. The common electrode CE is a patternless electrode entirely covering the second base substrate 210.

The second alignment layer 250 is disposed on the common electrode CE. The second alignment layer 250 is substantially the same with the first alignment layer 150 except for being disposed on the second base substrate 210. Thus, any repetitive descriptions will be omitted.

The second mesogen polymer layer 260 is disposed on the second alignment layer 250. The second mesogen polymer layer 260 is disposed on the second alignment layer 250 to fix the LC molecules so that the LC molecules have a pretilted angle adjacent to the second display substrate 200.

The LC layer 300 including the LC molecules is interposed between the first and second display substrates 100 and 200. Each of the LC molecules includes a compound including at least one carbon ring.

FIG. 6 and FIG. 7 are conceptual views illustrating a method of manufacturing the display panel in FIG. 5.

In FIG. 6 and FIG. 7, as shown in FIG. 5, layers of the first display substrate 100 formed under the first alignment layer 150 are conveniently omitted to be shown, and layers of the second display substrate 200 formed under the second alignment layer 250 are conveniently omitted to be shown.

Referring to FIG. 5 and FIG. 6, the gate line GL and the gate electrode GE are disposed on the first base substrate 110, and the first insulating layer 120 and the active pattern AP are sequentially formed. After the data line DL, the source electrode SE and the drain electrode DE are sequentially disposed on the first base substrate 110 on which the active pattern AP is formed, the second insulating layer 130, the organic layer 140 and the pixel electrode PE are sequentially formed.

The first alignment layer 150 are disposed on the first base substrate 110 on which the pixel electrode PE is formed, using a composition for forming an alignment layer. The composition may include an alignment polymer such as polyimide, polyamic acid, polysiloxane, polyvinylcinnamate, polyacrylate, polymethylmethacrylate, or the like. These may be used alone or a mixture thereof.

Although not shown in figures, in the first display substrate 100, the active pattern AP, the source electrode SE and the drain electrode DE may be formed by using a single mask. For example, a semiconductive layer and a source metal layer are sequentially disposed on the first insulating layer 120. A photo pattern including a first thickness portion and a second thickness portion thinner than the first thickness portion are disposed on the source metal layer. The semiconductive layer and the source metal layer may be patterned using the photo pattern as an etch stopping layer to form the active pattern AP, the source electrode SE and the drain electrode DE. An etched surface of the active pattern AP may be coincided with a sidewall surface of the source electrode SE and a sidewall surface of the drain electrode DE.

The light-blocking pattern 220, the color filter 230, the over-coating layer 240 and the common electrode CE are sequentially disposed on the second base substrate 210. The second alignment layer 250 is formed using a substantially same composition with the composition for forming the first alignment layer 150, on the second base substrate 210 on which the common electrode CE is formed.

An LC composition is interposed between the first display substrate 100 including the first alignment layer 150 and the second display substrate 200 including the second alignment layer 250. The LC composition includes LC molecules 310 and a reactive mesogen compound represented by Chemical Formula 1-1. The reactive mesogen compound is shown in FIG. 6 by a reference number “320.”

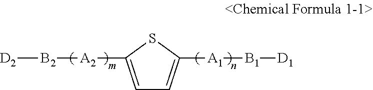

In Chemical Formula 1-1, each of A₁ and A₂ independently represents

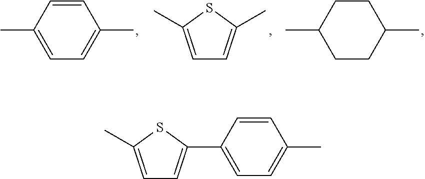

or a single bond, each of “n” and “m” independently represents an integer in a range of 0 to about 3 (“m” and “n” do not simultaneously represent 0), each of B₁ and B₂ independently represents a single bond or —(CH₂)ₖ— (“k” represents an integer in a range of 0 to 6), at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

The reactive mesogen compound 320 of the LC composition is substantially the same as the reactive mesogen compound illustrated above according to the present invention. Furthermore, the LC molecules 310 are substantially the same with the LC molecules in the LC composition illustrated above according to the present invention. Thus, any repetitive descriptions will be omitted.

The LC composition is interposed between the first and second alignment layers 150 and 250, and the LC molecules 310 and the reactive mesogen compound 320 are arranged having a predetermined alignment by the first and second alignment layers 150 and 250, in a non-electric state of the LC layer 300.

After the LC composition is disposed on one of the first alignment layer 150 and the second alignment layer 250, the first and second display substrates 100 and 200 are combined with each other so that the LC composition is interposed between the first and second display substrates 100 and 200. Alternatively, the LC composition may be injected between the first and second display substrates 100 and 200 combined with each other.

Referring to FIG. 7, after an electric field is formed between the first and second display substrates 100 and 200, a light is irradiated on the LC layer 300. For example, the light may be in the ultraviolet regime of the electromagnetic spectrum. For example, the light may be generated from a high-pressure mercury lamp having a maximal intensity in about 350 nm (nanometers) to about 370 nm. The electric field may be formed by applying voltages different from each other to the pixel electrode PE and the common electrode CE.

The LC molecules 310 are arranged having an alignment between the first and second alignment layers 150 and 250 by the electric field. Simultaneously, the reactive mesogen compound 320 is chemically reacted by the light. Thus, the first mesogen polymer layer 160 is disposed on the first alignment layer 150 by a polymerization of the reactive mesogen compounds 320. In addition, the second mesogen polymer layer 260 is disposed on the second alignment layer 250 by the polymerization of the reactive mesogen compounds 320.

Each of the first and second mesogen polymer layers 160 and 260 includes a polymer 322 as a product of the polymerization of the reactive mesogen compounds 320. The reactive mesogen compounds 320 may be polymerized by controlling the irradiation time to form the first and second mesogen polymer layers 160 and 260. The LC molecules 310 adjacent to the first display substrate 100 are arranged having a pretilted angle by the polymer 322, and the LC molecules 310 adjacent to the second display substrate 200 are arranged having a pretilted angle. Since the reactive mesogen compound includes thiophene, a polymerization time is decreased so that the time providing the light may be decreased compared to a conventional reactive mesogen compound, although the light is provided using the high-pressure mercury lamp as a light source. The light is provided to the LC layer 300 so that an amount of the reactive mesogen compounds 320 remained in the LC layer 300 may be minimized.

Then, the voltages applied to the pixel electrode PE and the common electrode CE are changed so that the LC layer 300 is in a non-electric state. Although the LC layer 300 is in the non-electric state, the LC molecules 310 adjacent to each of the first and second display substrates 100 and 200 are oriented by a predetermined angle by the first and second mesogen polymer layers 160 and 260. The LC molecules are stable in this position. Thus, the LC molecules 310 are in a pretilted angle in the non-electric state.

In addition, an ultraviolet ray is provided to the LC layer 300 in the non-electric state in order to maximally polymerize the reactive mesogen compounds 320 remained in the LC layer 300 that are not polymerized in the electric state.

According to the above descriptions, the reactive mesogen compound according to the present invention includes thiophene so that the reactive mesogen compound may be activated by a light having a wavelength in a range of about 300 nm to about 700 nm and may have a maximal absorption in a wave length between about 300 nm and about 370 nm. Therefore, photosensitivity in the wavelength may be maximized so that the process time for forming the first and second mesogen polymer layers 160 and 260 are reduced. In addition, an amount of the reactive mesogen compounds 320 remained in the LC layer 300 of the display panel 500 as a final product may be minimized, so that an effect of controlling the LC molecules 310 by the reactive mesogen compounds 320 may be minimized when the display panel 500 displays an image.

Hereinafter, a method of manufacturing the display panel 500 shown in FIG. 4 and FIG. 5 using a composition for an alignment layer according to the present invention illustrated above will be described.

FIG. 8 is a flow chart illustrating another method of manufacturing the display panel in FIG. 5.

Referring to FIG. 8 with FIG. 5, the first alignment layer 150 is formed using a composition for forming an alignment layer different from the composition illustrated in FIG. 6 and FIG. 7 on the first base substrate 110 on which the pixel electrode PE is formed (Step S610).

The composition, differently illustrated in FIG. 6 and FIG. 7, includes an alignment polymer having a functional group represented by Chemical Formula 1-2.

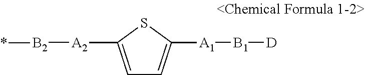

In Chemical Formula 1-2, each of each of A₁ and A₂ independently represents

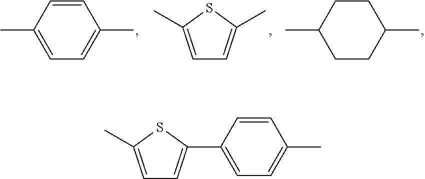

or a single bond, each of “n” and “m” independently represents an integer in a range of 0 to about 3 (“m” and “n” do not simultaneously represent 0), each of B₁ and B₂ independently represents a single bond or —(CH₂)ₖ— (“k” represents an integer in a range of 0 to about 6), at least one —(CH₂)— in each of B₁ and B₂ is replaceable with —O—, —COO—, —OCO— or —CO—, D independently represents methacrylate or acrylate, and at least one hydrogen atom of each of A₁, A₂ and 2,5-thiophene is replaceable with F, Cl, OCH₃ or an alkyl group having a carbon atom of 1 to about 6.

The composition is coated on the first base substrate 110 on which the pixel electrode PE is formed to form the first alignment layer 150. A main chain of the alignment polymer forms a base of the first alignment layer 150 as a layer having a predetermined thickness, and the functional group represented by Chemical Formula 1-2 may be protruded to a surface of the layer formed by the main chain or may be disposed in the layer.

Similarly, the composition used in forming the first alignment layer 150 is coated on the second base substrate 210 on which the common electrode CE is formed to form the second alignment layer 250 (Step S620).

Then, the LC layer 300 is formed between the first display substrate 100 including the first alignment layer 150 and the second display substrate 200 including the second alignment layer 250. The LC layer 300 is formed using a LC composition including the LC molecules 310 (Step S630). The LC layer 300 may include the LC molecules 310 without the reactive mesogen compounds 320, differently shown in FIG. 6.

After forming the LC layer 300, an electric field is formed between the first display substrate 100 and the second display substrate 200 and a light is provided to the LC layer 300. Thus, the first and second mesogen polymer layers 160 and 260 are formed (Step S640).

The functional group disposed to a surface of the first alignment layer 150 is polymerized by the light to form the first mesogen polymer layer 160. In addition, the functional group disposed on a surface of the second alignment layer 250 is polymerized by the light to form the second mesogen polymer layer 260. The LC molecules 320 may have a pretilted angle by the first and second mesogen polymer layers 160 and 260 although the LC layer 300 is the non-electric state.

The first and second mesogen polymer layers 160 and 260 are formed, and thus the reactive mesogen compounds of the first and second alignment layers 150 and 250 before providing the light are removed. The reactive mesogen compounds include thiophene to decrease a photo-reaction time of an UV ray. Therefore, a process time of manufacturing the display panel 500 may be significantly reduced and an amount of the reactive mesogen compounds used in the process may also be minimized.

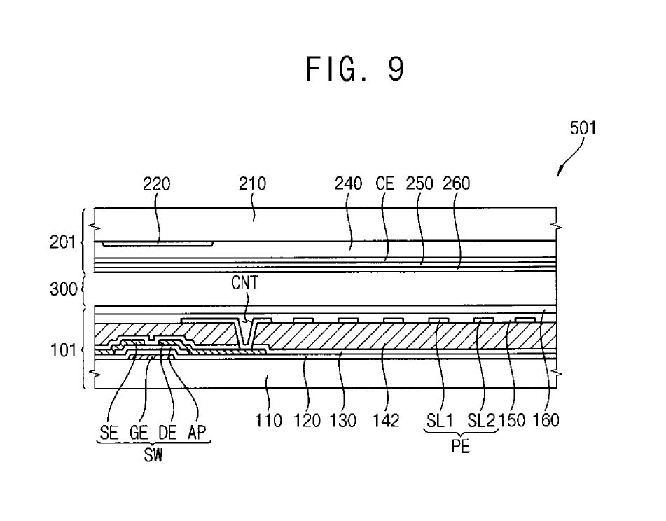

FIG. 9 is a cross-sectional view illustrating a display panel manufactured according to still another example of the present invention.

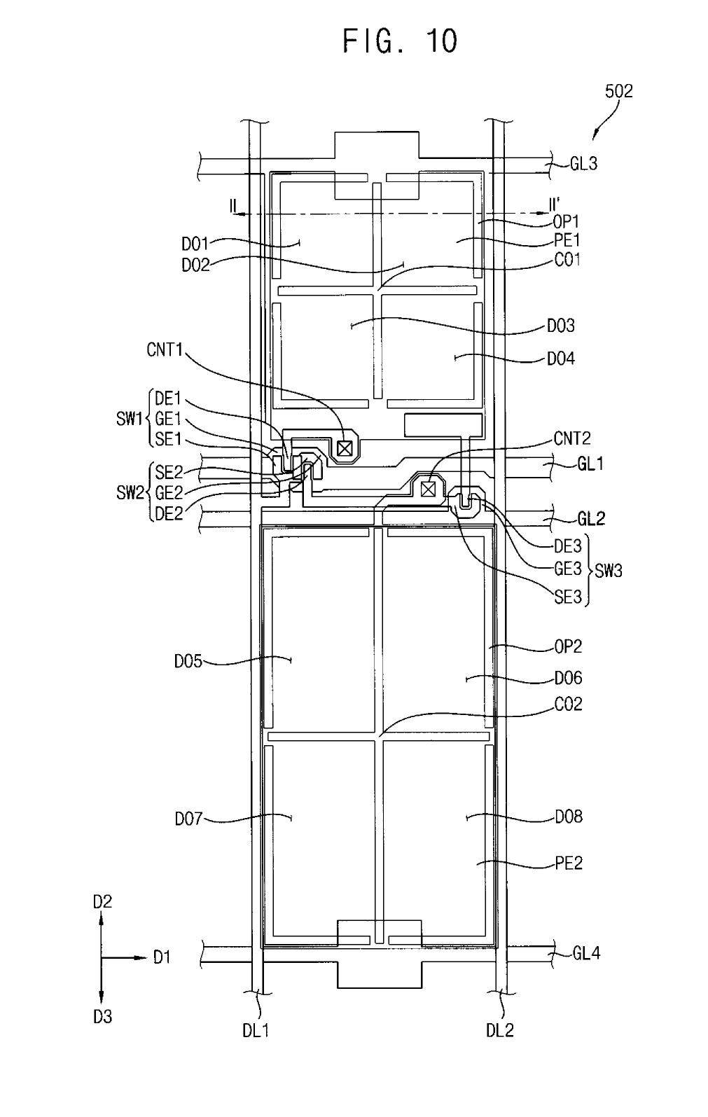

Referring to FIG. 9, a display panel 501 includes a first display substrate 101, a second display substrate 201 facing the first display substrate 101, and an LC layer 300. A plan view of the display panel 501 in FIG. 9 is substantially the same with a plan view of the display panel 500 in FIG. 4, and thus the display panel 501 will be illustrated with reference to FIG. 9 and FIG. 10.

The first display substrate 101 includes a gate line GL disposed on a first base substrate 110, a first insulating layer 120, a data line DL, a switching element SW, a second insulating layer 130, a color filter layer 142, a pixel electrode PE, a first alignment layer 150 and a first mesogen polymer layer 160. The first display substrate 101 is substantially the same with the first display substrate 100 in FIG. 5 except including the color filter layer 142 in lieu of the organic layer 140, and thus any repetitive description will be omitted. Although not shown in figures, the organic layer 140 of the first display substrate 100 in FIG. 5 may be disposed between the color filter layer 142 and the second insulating layer 130.

The second display substrate 201 includes a light-blocking pattern 220 disposed on a second base substrate 210, an over-coating layer 240, a second alignment layer 250 and a second mesogen polymer layer 260. The second display substrate 201 is substantially the same with the second display substrate 200 in FIG. 5 except for omitting the color filter 230, and thus any repetitive descriptions will be omitted.

The LC layer 300 includes a plurality of LC molecules interposed between the first mesogen polymer layer 160 and the second mesogen polymer layer 260. The LC molecules are slanted having a predetermined pretilted angle that is determined by the first and second mesogen polymer layers 160 and 260 without an electric field.

Hereinafter, a method of manufacturing the display panel 501 shown in FIG. 9 will be illustrated referring to FIG. 9 with FIG. 6 and FIG. 7.

Firstly, the gate electrode GE is disposed on the first base substrate 110, and the first insulating layer 120 and the active pattern AP are formed. After forming the source electrode SE and the drain electrode DE on the first base substrate 110 on which the active pattern AP is formed, the second insulating layer 130 and the color filter layer 142 and the pixel electrode PE are formed sequentially.

The first alignment layer 150 is formed using the composition for forming an alignment layer on the first base substrate 110 on which the pixel electrode PE is formed. The composition for forming the alignment layer may include an alignment polymer including polyimide, polyamic acid, polysiloxane, polyvinylcinnamate, polyacrylate, polymethylmethacrylate, or the like. These may be used alone or in a mixture thereof.

The light-blocking pattern 220, the over-coating layer 240 and the common electrode are sequentially disposed on the second base substrate 210. The second alignment layer 250 is formed using the composition for forming the first alignment layer 150 on the second base substrate 210 on which the common electrode CE is formed.

An LC composition is interposed between the first display substrate 101 including the first alignment layer 150 and the second display substrate 201 including the second alignment layer 250. The LC composition includes LC molecules generally used in an LC composition and a reactive mesogen compound. The reactive mesogen compound used in the LC composition is substantially the same with the reactive mesogen compound illustrated above according to the present invention. In addition, the LC molecules are substantially the same with the LC molecules of the LC composition illustrated above according to the present invention. Thus, any repetitive descriptions will be omitted.

Then, a process forming the first and second mesogen polymer layers 160 and 260 using the reactive mesogen compound is substantially the same with the process illustrated in FIG. 6 and FIG. 7. Thus, any repetitive descriptions will be omitted.

In the display panel 501 shown in FIG. 9, the first alignment layer 150 and the first mesogen polymer layer 160 are formed by substantially the same method illustrated in the FIG. 8 using a composition for forming an alignment layer including an alignment polymer having a functional group which includes thiophene as a side chain. In addition, the second alignment layer 250 and the second mesogen polymer layer 260 are formed using the composition for forming the alignment layer.

Although not shown in figures, in a display panel having a structure where the light-blocking pattern 220 is disposed on the first base substrate 110, and not the second base substrate 210, the display panel may be manufactured by substantially the same method with illustrated in FIG. 6 and FIG. 7 using the LC composition including the reactive mesogen compound according to the present invention or a method illustrated in FIG. 8.

In addition, a display panel having a structure which the light-blocking pattern 220 is disposed on the first base substrate 110, the display panel may be manufactured by substantially the same method with illustrated in FIG. 6 and FIG. 7 using the LC composition including the reactive mesogen compound according to the present invention or a method illustrated in FIG. 8.

FIG. 10 is a plane view illustrating a display panel manufactured according to further still another example of the present invention.

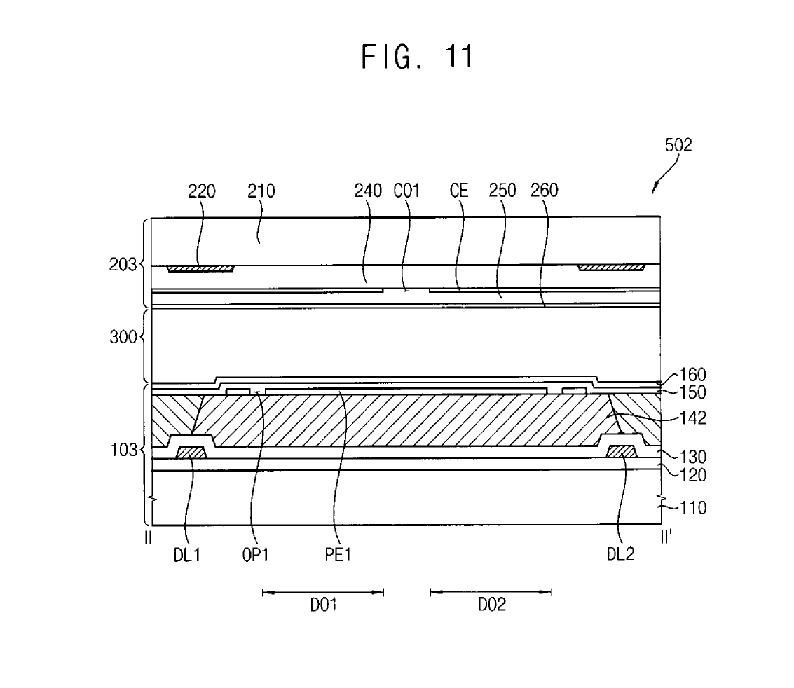

FIG. 11 is a cross-sectional view taken along a line II-II′ in FIG. 10.

Referring to FIG. 10 and FIG. 11, a display panel 502 includes a first display substrate 103, a second display substrate 203 and an LC layer 300. The LC layer 300 may include an LC composition having a negative dielectric anisotropy.

The first display substrate 103 includes gate lines GL1, GL2, GL3 and GL4 disposed on a first base substrate 110, a first data line DL1, a second data line DL2, a first switching element SW1, a first switching element SW2, a third switching element SW3, a color filter layer 142, a first pixel electrode PE1, a second pixel electrode PE2, a first alignment layer 150 and a first mesogen polymer layer 160.

The gate lines GL1, GL2, GL3 and GL4 extend in a first direction D1 and include a first gate line GL1 and a second gate line GL2 adjacent to each other, a third gate line GL3 disposed in a second direction D2, and a fourth gate line GL4 disposed in a third direction D3. Each of the second and third directions crosses the first direction D1 and extends a reverse direction to each other. The first to fourth gate lines GL1, GL2, GL3 and GL4 may be insulated from the first and second data lines DL1 and DL2 by a first insulating layer 120.

The first switching element SW1 includes a first gate line GE1 connected to the first gate line GL1, a first source electrode SE connected to the first data line DL1, and a first drain electrode DE1 spaced apart from the first source electrode SE. The first drain electrode DE1 is connected to the first pixel electrode PE through a first contact hole CNT1.

The second switching element SW2 includes a second gate electrode GE2 connected to the first gate line GL1, a second source electrode SE2 connected to the first data line DL1, a second drain electrode DE2 spaced apart from the second source electrode SE. The second source electrode SE2 is connected to the first source electrode SE1. The second drain electrode DE is connected to the second pixel electrode PE2 through a second contact hole CNT2.

The third switching element SW3 includes a third gate electrode GE3 connected to the second gate line GL2, a third source electrode SE3 connected to the first data line DL1 and the second drain electrode DE2, and a third drain electrode DE3 spaced apart from the third source electrode SE3. The third drain electrode DE3 overlaps with the first pixel electrode PE1. A portion overlapping with the third drain electrode DE3 and the first pixel electrode PE1 defines to a down-capacitor.

Although not shown in figures, each of the first, second and third switching elements SW1, SW2 and SW3 includes a semiconductive pattern.

The first switching element SW1 and the second switching element SW2 are turned on corresponding to a first gate signal applied to the first gate line GL1, and the third switching element SW3 is turned on corresponding to a second gate signal applied to the second gate line GL2. Thus, when the third switching element SW3 is turned on, the down-capacitor decreases a data voltage charged by the second pixel electrode PE2. That is, a region in which the first pixel electrode PE is formed may be defined to a high-pixel of the display panel 502 and a region in which the second pixel electrode PE2 is formed may be defined to a low-pixel.

The color filter layer 142 is disposed on the first to third switching elements SW1, SW2 and SW3. The first and second pixel electrodes PE1 and PE2 are disposed on the color filter layer 142.

The first pixel electrode PE1 includes first opening portions OP1 formed along a shape of the first pixel electrode PE1. For example, when the first pixel electrode PE1 has a quadrangle shape, four first opening portions OP1 formed along two side portion crossing each other is disposed in each of four edge portions. When the first opening portions OP1 are connected to each other, a shape connected to the first opening portions OP1 may be substantially the same with or similar to that of the first pixel electrode PE1, and each of the first opening portions OP1 is spaced apart from each other.

The second pixel electrode PE2 includes second opening portions OP2 formed along a shape of the second pixel electrode PE2. A arrangement structure of the second opening portions OP2 in the second pixel electrode PE2 is substantially the same as that of the first opening portions OP1 in the first pixel electrode PE1, and thus any repetitive descriptions will be omitted.

The first alignment layer 150 and the first mesogen polymer layer 160 are disposed on the first and second pixel electrodes PE1 and PE2. The first alignment layer 150 and the first mesogen polymer layer 160 may be substantially the same with illustrated in FIG. 5. Thus, any repetitive descriptions will be omitted. The first alignment layer 150 may be a vertical alignment layer.

The second display substrate 203 includes a light-blocking pattern 220 disposed on a second base substrate 210, an over-coating layer 240, a common electrode CE a second alignment layer 250 and a second mesogen polymer layer 260. The second display substrate 203 is substantially the same with the second display substrate 201 illustrated in FIG. 9 except for the common electrode CE. Thus, any repetitive descriptions will be omitted. The second alignment layer 250 may be a vertical alignment layer.

The common electrode CE is entirely disposed on the second base substrate 210 and includes a third opening portion CO1 and a fourth opening portion CO2. Each of the third and fourth opening portions CO1 and CO2 may have a cross shape. The third opening portion CO1 overlaps with the first pixel electrode PE1 and the fourth opening portion CO2 overlaps with the second pixel electrode PE2.

When viewed in a plan, with respect to a central portion of the third opening portion CO1, each of the first opening portions OP1 may be disposed in two, five, seven and eleven o'clock directions of the third opening portion CO1. In addition, with respect to a central portion of the fourth opening portion CO2, each of the second opening portions OP2 may be disposed in two, five, seven and eleven o'clock directions of the fourth opening portion CO2. In FIG. 10, the fourth opening portion CO2 has a single cross shape. Alternatively, the fourth opening portion CO2 may have a shape connected to at least two crosses.

The first pixel electrode PE1 and the third opening portion CO1 of the common electrode CE may define to a first domain region DO1, a second domain region DO2, a third domain region DO3 and a fourth domain region DO4. That is, the high-pixel may be divided to the first to fourth domain regions DO1, DO2, DO3 and DO4. In addition, the second pixel electrode PE2 and the fourth opening portion CO2 of the common electrode CE may define to a fifth domain region DO5, a sixth domain region DO6, a seventh domain region DO7 and a eighth domain region DO8. That is, the low-pixel may be divided to the fifth to eighth domain regions DO5, DO6, DO7 and DO8. The first to eighth domain regions DO1 to DO8 defined by the first and second pixel electrodes PE1 and PE2 and the common electrode CE form domains of LC molecules of the LC 300 to improve a viewing angle of the display panel 502.

Hereinafter, a method of manufacturing the display panel 502 will be illustrated referring to FIG. 11.

Firstly, the first to fourth gate lines GL1, GL2, GL3 and GL4 and the first to third gate electrodes GE1, GE2 and GE3 are disposed on the first base substrate 110, and the first insulating layer 120 is formed thereon.

The semiconductive pattern is disposed on the first base substrate 110 on which the first insulating layer 120 is formed, and the first to third source electrodes SE1, SE2 and SE3, the first to third drain electrodes DE1, DE2 and DE3, the first and second data lines DL1 and DL2 are disposed on the first base substrate 110 on which the semiconductive pattern is formed. Alternatively, the semiconductive pattern may be formed in forming the first to third source electrodes SE1, SE2 and SE3, the first to third drain electrodes DE1, DE2 and DE3, the first and second data lines DL1 and DL2. A dummy pattern including substantially the same layer with the semiconductive pattern may be formed between the first and second data lines DL1 and DL2 and the first insulating layer 120.

A second insulating layer 130 is disposed on the first base substrate 110 on which the first to third source electrodes SE1, SE2 and SE3, the first to third drain electrodes DE1, DE2 and DE3, the first and second data lines DL1 and DL2. The color filter layer 142 is disposed on the second insulating layer 130.

The first and second pixel electrodes PE1 and PE2 are disposed on the color filter layer 142.

Then, the first alignment layer 150 is disposed on the first base substrate 110 on which the first and second pixel electrodes PE1 and PE2 are formed. The first alignment layer 150 may be formed using a general composition for forming an alignment layer including an alignment polymer.

After sequentially forming the light-blocking pattern 220, the over-coating layer 240 and the common electrode CE on the second base substrate 210, the second alignment layer 250 is disposed on the second base substrate 210 on which the common electrode CE is formed. The second alignment layer 250 may be formed using the composition used in forming the first alignment layer 150.

Then, an LC composition is interposed between the first base substrate 110 on which the first alignment layer 150 is formed and the second base substrate 210 on which the second alignment layer 250. The LC composition may include a plurality of LC molecules and a reactive mesogen compound. The LC composition may have a negative dielectric anisotropy. The reactive mesogen compound is substantially the same with the reactive mesogen compound represented by Chemical Formula 1-1 according to the present invention, and thus any repetitive descriptions will be omitted.

A light is irradiated on to the LC composition disposed between the first and second base substrates 110 and 210 from outside of the first base substrate 110 or outside of the second base substrate 210 to form the first mesogen polymer layer 160 on the first alignment layer 150 and to form the second mesogen polymer layer 260 on the second alignment layer 250.

Alternatively, in the display panel 502 in FIG. 10 and FIG. 11, each of the first and second alignment layers 150 and 250 is formed using an alignment polymer including a functional group reacted by light. The alignment polymer may include the functional group represented by Chemical Formula 1-2 as a side chain, and the alignment polymer is substantially the same with the composition for forming the alignment layer illustrated above, and thus any repetitive descriptions will be omitted.

The LC layer 300 is formed using a general LC composition not including the reactive mesogen compound, and a light is provided to the first and second alignment layers 150 and 250 with already forming the LC layer 300. Thus, the side chain of the alignment polymer substantially the same with illustrated in FIG. 8 is reacted with the light to form the first mesogen polymer layer 160 on the first alignment layer 150 and to form the second mesogen polymer layer 260 on the second alignment layer 250.

Thus, the display panel 502 shown in FIG. 10 and FIG. 11 may be manufactured. The LC molecules of the LC composition may have a pretitled angle by the first and second mesogen polymer layers 160 and 260 to improve a response rate of the display panel 502.

FIG. 10 and FIG. 11 show that the color filter layer 142 is disposed on the first base substrate 110. Alternatively, the color filter layer 142 may be disposed on the second base substrate 210 such as the second display substrate 200 in FIG. 5. In addition, the light-blocking pattern 220 disposed on the second base substrate 210 in FIG. 10 and FIG. 11 may be disposed on the first base substrate 110.

In addition, although not shown in figures, an organic layer 140 in FIG. 5 may be further formed between the first and second pixel electrodes PE1 and PE2 and the second insulating layer 140 in the first display substrate 103.

As described above in detail, although using a glass substrate as a base substrate, the photo-reactive efficiency of the reactive mesogen compound may be improved in a process by irradiating the reactive mesogen compound with light having greater wavelength than the wavelength absorbed by the glass substrate. That is, the reactive mesogen compound is activated by a light having a wavelength in a range of about 300 nm to about 700 nm, and the intensity of the reactive mesogen compound for light having a wavelength between about 300 nm and about 370 nm may be maximized. Thus, a polymerization time of the reactive mesogen compound is decreased so that an entire manufacturing time may be decreased to improve the productivity. In addition, the reactive mesogen compound is reacted with the light so that an amount of the reactive mesogen compound remaining in an LC layer is minimized to improve the reliability of the manufacturing process.

It will be understood that when an element is referred to as being “on” another element, it can be directly on the other element or intervening elements may be present therebetween. In contrast, when an element is referred to as being “directly on” another element, there are no intervening elements present. As used herein, the term “and/or” includes any and all combinations of one or more of the associated listed items.

It will be understood that, although the terms “first,” “second,” “third” etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, “a first element,” “component,” “region,” “layer” or “section” discussed below could be termed a second element, component, region, layer or section without departing from the teachings herein.

The terminology used herein is for the purpose of describing particular embodiments only and is not intended to be limiting. As used herein, the singular forms “a,” “an,” and “the” are intended to include the plural forms, including “at least one,” unless the content clearly indicates otherwise. “Or” means “and/or.” It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Furthermore, relative terms, such as “lower” or “bottom” and “upper” or “top,” may be used herein to describe one element's relationship to another element as illustrated in the Figures. It will be understood that relative terms are intended to encompass different orientations of the device in addition to the orientation depicted in the Figures. For example, if the device in one of the figures is turned over, elements described as being on the “lower” side of other elements would then be oriented on “upper” sides of the other elements. The exemplary term “lower,” can therefore, encompasses both an orientation of “lower” and “upper,” depending on the particular orientation of the figure. Similarly, if the device in one of the figures is turned over, elements described as “below” or “beneath” other elements would then be oriented “above” the other elements. The exemplary terms “below” or “beneath” can, therefore, encompass both an orientation of above and below.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Exemplary embodiments are described herein with reference to cross section illustrations that are schematic illustrations of idealized embodiments. As such, variations from the shapes of the illustrations as a result, for example, of manufacturing techniques and/or tolerances, are to be expected. Thus, embodiments described herein should not be construed as limited to the particular shapes of regions as illustrated herein but are to include deviations in shapes that result, for example, from manufacturing. For example, a region illustrated or described as flat may, typically, have rough and/or nonlinear features. Moreover, sharp angles that are illustrated may be rounded. Thus, the regions illustrated in the figures are schematic in nature and their shapes are not intended to illustrate the precise shape of a region and are not intended to limit the scope of the present claims.

The foregoing is illustrative of the present invention and is not to be construed as limiting thereof. Although a few example embodiments of the present invention have been described, those skilled in the art will readily appreciate that many modifications are possible in the example embodiments without materially departing from the novel teachings and advantages of the present invention. Accordingly, all such modifications are intended to be included within the scope of the present invention as defined in the claims. In the claims, means-plus-function clauses are intended to cover the structures described herein as performing the recited function and not only structural equivalents but also equivalent structures. Therefore, it is to be understood that the foregoing is illustrative of the present invention and is not to be construed as limited to the specific example embodiments disclosed, and that modifications to the disclosed example embodiments, as well as other example embodiments, are intended to be included within the scope of the appended claims. The present invention is defined by the following claims, with equivalents of the claims to be included therein.

What is claimed is:

1. A reactive mesogen compound represented by Chemical Formula 1-1; 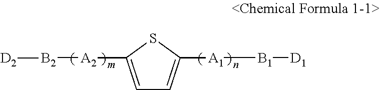
wherein in Chemical Formula 1-1, each of A₁ and A₂ independently represents 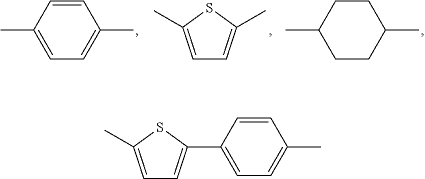
or a single bond, each of “n” and “m” independently represents an integer in a range of 0 to about 3 and where “m” and “n” do not simultaneously represent 0, each of B₁ and B₂ independently represents a single bond or —(CH₂)ₖ— where “k” represents an integer in a range of 0 to about 6, at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

2. The reactive mesogen compound of claim 1, wherein the reactive mesogen compound is selected from the group consisting of Chemical Formula 2-1, Chemical Formula 2-2 and Chemical Formula 2-3, 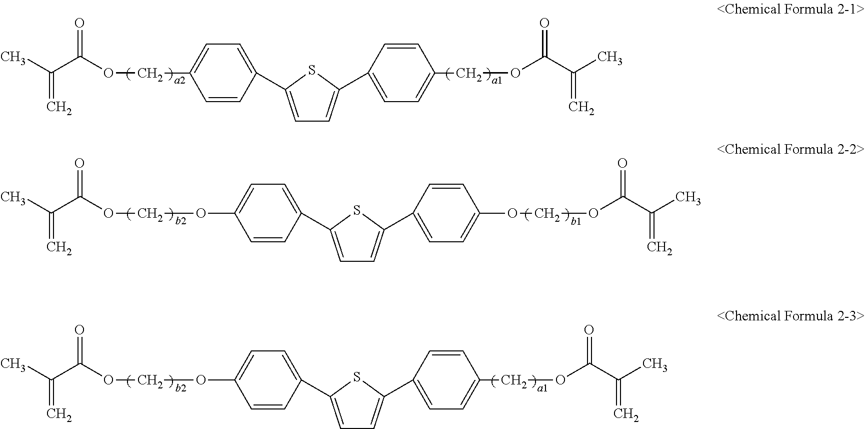
wherein each of a1 and a2 independently represents an integer in a range of 0 to about 6, and each of b1 and b2 independently represents an integer in a range of 1 to about 5.

3. The reactive mesogen compound of claim 1, wherein the reactive mesogen compound is selected from the group consisting of Chemical Formula 3-1, Chemical Formula 3-2, Chemical Formula 3-3 and Chemical Formula 3-4, 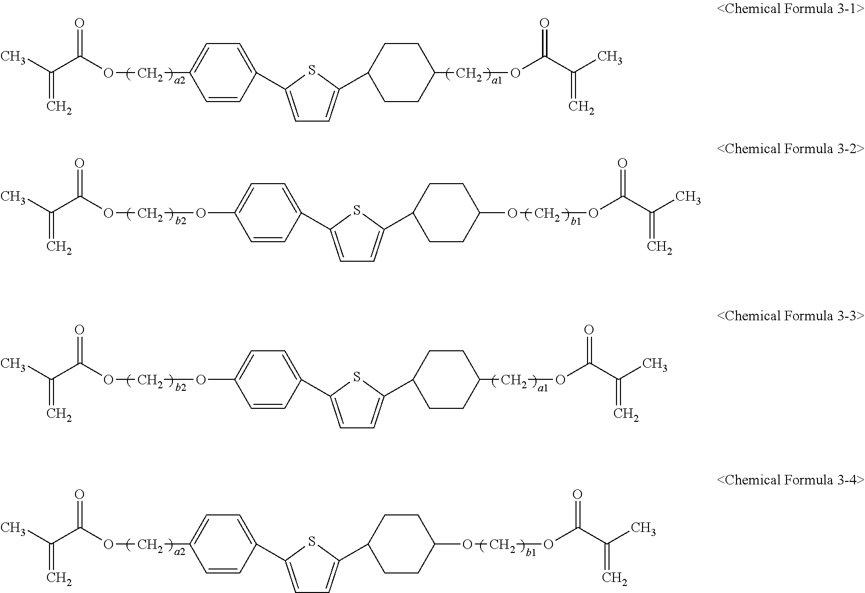
wherein each of a1 and a2 independently represents an integer in a range of 0 to about 6, and each of b1 and b2 independently represents an integer in a range of 1 to about 5.

4. The reactive mesogen compound of claim 1, wherein the reactive mesogen compound is selected from the group consisting of Chemical Formula 4-1, Chemical Formula 4-2, Chemical Formula 4-3 and Chemical Formula 4-4, 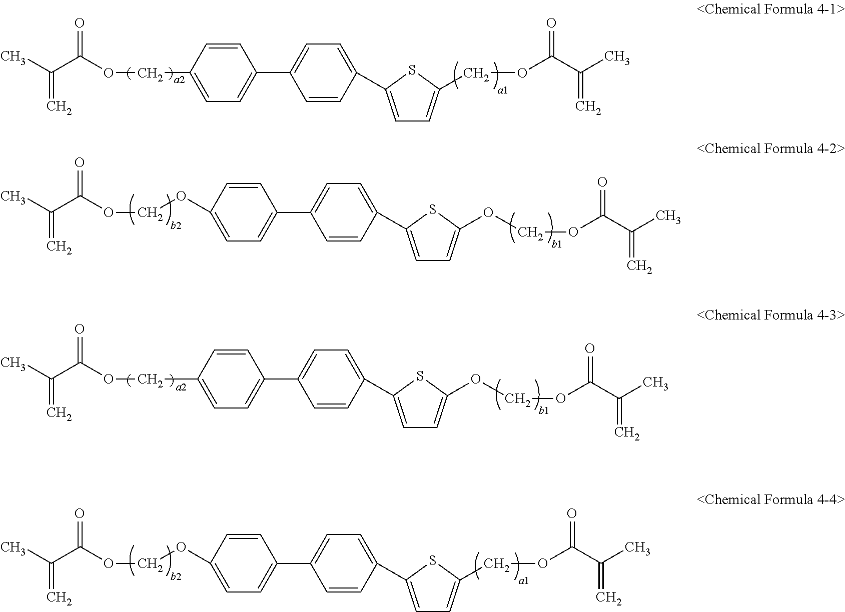
wherein each of a1 and a2 independently represents an integer in a range of 0 to about 6, and each of b1 and b2 independently represents an integer in a range of 1 to about 5.

5. The reactive mesogen compound of claim 1, wherein the reactive mesogen compound is selected from the group consisting of Chemical Formula 5-1, Chemical Formula 5-2, Chemical Formula 5-3 and Chemical Formula 5-4, 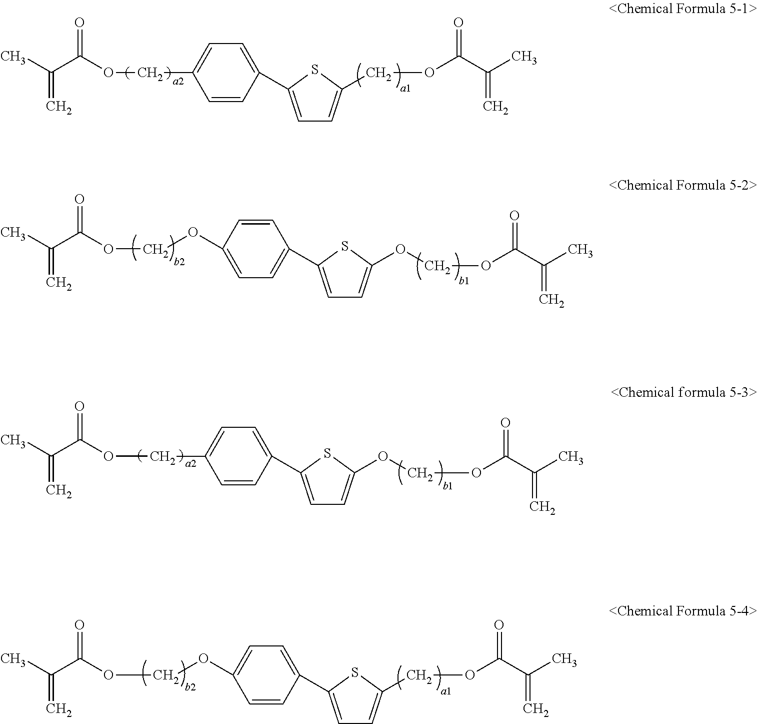
wherein each of a1 and a2 independently represents an integer in a range of 0 to about 6, and each of b1 and b2 independently represents an integer in a range of 1 to about 5.

6. The reactive mesogen compound of claim 1, wherein the reactive mesogen compound is selected from the group consisting of Chemical Formula 6-1, Chemical Formula 6-2, Chemical Formula 6-3 and Chemical Formula 6-4, 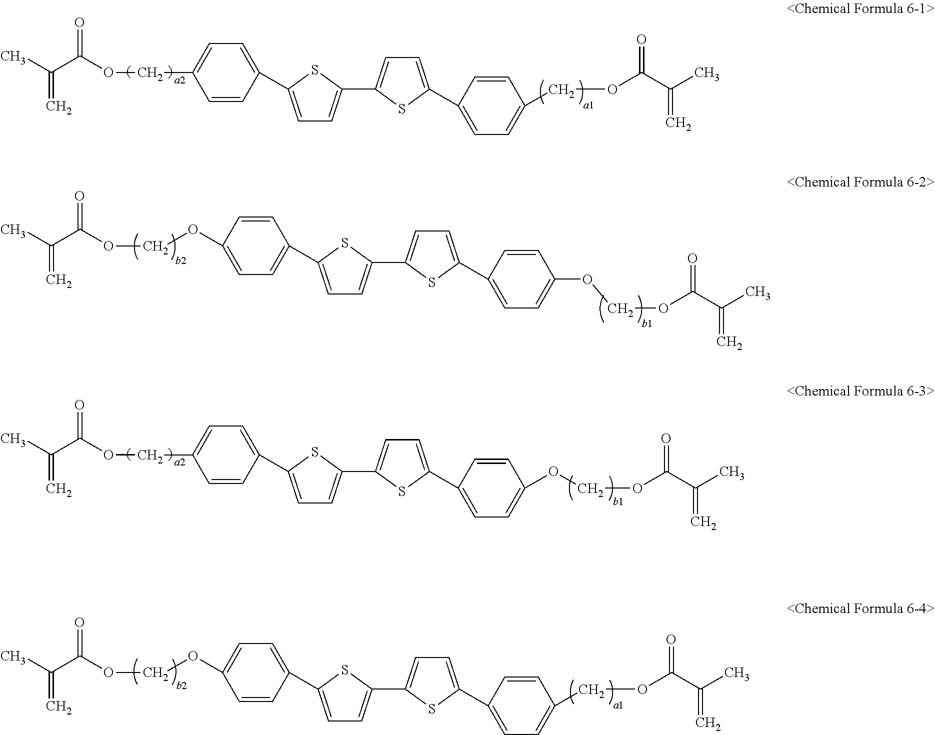
wherein each of a1 and a2 independently represents an integer in a range of 0 to about 6, and each of b1 and b2 independently represents an integer in a range of 1 to about 5.

7. The reactive mesogen compound of claim 1, wherein the reactive mesogen compound is activated by light having a wavelength between about 300 nm and about 700 nm.

8. A liquid crystal composition comprising:
liquid crystal molecules including a compound having at least one carbon ring; and a reactive mesogen compound represented by Chemical Formula 1-1; 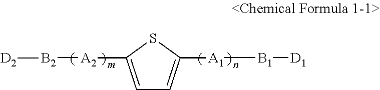
wherein in Chemical Formula 1-1, each of A₁ and A₂ independently represents 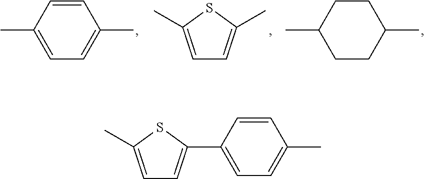
or a single bond, each of “n” and “m” independently represents an integer in a range of 0 to about 3; “m” and “n” do not simultaneously represent 0, each of B₁ and B₂ independently represents a single bond or —(CH₂)ₖ—; where “k” represents an integer in a range of 0 to 6, at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

9. The liquid crystal composition of claim 8, wherein an amount of the liquid crystal molecules is about 95% by weight to about 99.9% by weight based on a total weight of the liquid crystal composition, an amount of the reactive mesogen compound is about 0.1% by weight to about 5% by weight, based on a total weight of the liquid crystal composition.

10. The liquid crystal composition of claim 8, wherein the reactive mesogen compound is activated by a light having a wavelength between about 300 nm and about 700 nm.

11. A method of manufacturing a display panel, the method comprising:
forming a first display substrate which includes a pixel electrode and a first alignment layer disposed on the pixel electrode;
forming a second display substrate which faces the first display substrate and includes a second alignment layer;
interposing a liquid crystal composition between the first and second display substrates, the liquid crystal composition including liquid crystal molecules having at least one carbon ring and a reactive mesogen compound represented by Chemical Formula 1-1; and
irradiating a light on to the liquid crystal composition with the liquid crystal molecules pretilted by an electric field between the first and second display substrates; 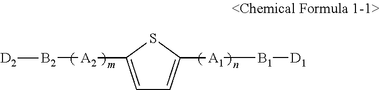
wherein in Chemical Formula 1-1, each of A₁ and A₂ independently represents 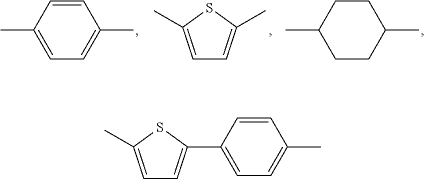
or a single bond, each of “n” and “m” independently represents an integer in a range of 0 to about 3; “m” and “n” do not simultaneously represent 0, each of B₁ and B₂ independently represents a single bond or —(CH₂)ₖ—; wherein “k” represents an integer in a range of 0 to about 6, at least one —(CH₂)— in each of B₁ and B₂ is replaceable with —O—, —COO—, —OCO— or —CO—, each of D₁ and D₂ independently represents methacrylate or acrylate, and at least one hydrogen atom of each of A₁, A₂ and 2,5-thiophene is replaceable with F, Cl, OCH₃ or an alkyl group having 1 to about 6 carbon atoms.

12. The method of claim 11, further comprising irradiating light on to the liquid crystal composition in a non-electric state between the first and second display substrates after irradiating the light on to the liquid crystal composition.

13. The method of claim 11, wherein the light has a wavelength in a range of about 300 nm to about 700 nm.

14. The method of claim 11, wherein an amount of the liquid crystal molecules is about 95% by weight to about 99.9% by weight, based on a total weight of the liquid crystal composition, and an amount of the reactive mesogen compound is about 0.1% by weight to about 5% by weight, based on a total weight of the liquid crystal composition.

15. The method of claim 11, wherein the reactive mesogen compound is represented by Chemical Formula 2-1, 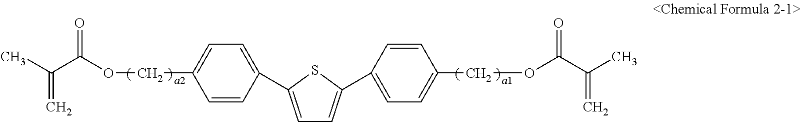
wherein each of a1 and a2 independently represents an integer in a range of 0 to 6.

16. The method of claim 11, wherein the providing the light to the liquid crystal composition comprises:
reacting the reactive mesogen compound upon irradiation with light to form a first mesogen polymer layer on the first alignment layer, the first mesogen polymer layer fixing the liquid crystal molecules disposed adjacent to the first display substrate so that the liquid crystal molecules have a pretilted angle on a surface of the first mesogen polymer layer; and
reacting the reactive mesogen compound upon irradiation with the light to form a second mesogen polymer layer on the second alignment layer, the second mesogen polymer layer fixing the liquid crystal molecules disposed adjacent to the second display substrate so that the liquid crystal molecules have a pretilted angle on a surface of the second mesogen polymer layer.

17. A method of manufacturing a display panel, the method comprising:
forming a first alignment layer on a pixel electrode of a first display substrate using a composition for manufacturing an alignment layer, the composition including an alignment polymer having a functional group represented by Chemical Formula 1-2;
forming a second alignment layer of a second display substrate facing the first display substrate using the composition;
interposing a liquid crystal composition between the first and second display substrates; and
providing a light to the first and second display substrates, between which an electric field is formed, to cause a photo-reaction of the functional group of the first and second alignment layers, 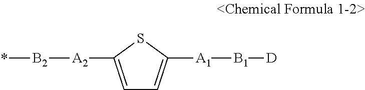
wherein in Chemical Formula 1-2, each of each of A₁ and A₂ independently represents 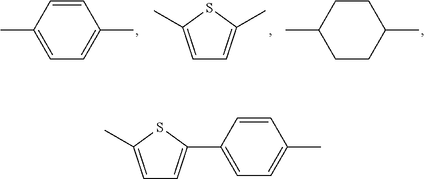
or a single bond, each of “n” and “m” independently represents an integer in a range of 0 to about 3; “m” and “n” do not simultaneously represent 0, each of B₁ and B₂ independently represents a single bond or —(CH₂)ₖ—; where “k” represents an integer in a range of 0 to about 6, at least one —(CH₂)— in each of B₁ and B₂ is replaceable with —O—, —COO—, —OCO— or —CO—, D independently represents methacrylate or acrylate, and at least one hydrogen atom of each of A₁, A₂ and 2,5-thiophene is replaceable with F, Cl, OCH₃ or an alkyl group having 1 to about 6 carbon atoms.

18. The method of claim 17, wherein providing the light comprises:
forming a first mesogen polymer layer on the first alignment layer, the first mesogen polymer layer fixing the liquid crystal molecules disposed adjacent to the first display substrate so that the liquid crystal molecules have a pretilted angle on a surface of the first mesogen polymer layer; and
forming a second mesogen polymer layer on the second alignment layer, the second mesogen polymer fixing the liquid crystal molecules disposed adjacent to the second display substrate so that the liquid crystal molecules have a pretilted angle on a surface of the second mesogen polymer layer.

19. The method of claim 17, wherein a main chain combined with the functional group in the alignment polymer comprises at least one selected from the group consisting of polyimide, polyamic acid, polysiloxane, polyvinylcinnamate, polyacrylate and polymethylmethacrylate.

20. A display panel comprising:
a first display substrate including a pixel electrode, a first alignment layer disposed on the pixel electrode and a first mesogen polymer layer disposed on the first alignment layer;
a second display substrate facing the first display substrate and including a second alignment layer and a second mesogen polymer layer disposed on the second alignment layer; and
a liquid crystal layer interposed between the first and second mesogen polymer layers,
wherein each of the first and second mesogen polymer layers is formed by reacting a functional group represented by Chemical Formula 1-2 with a light; 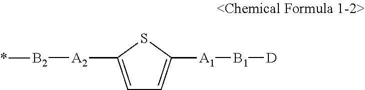
wherein in Chemical Formula 1-2, each of each of A₁ and A₂ independently represents 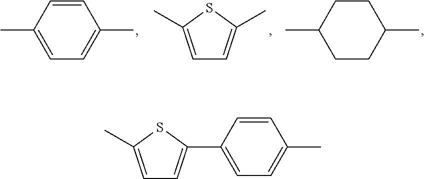
or a single bond, each of “n” and “m” independently represents an integer in a range of 0 to about 3; “m” and “n” do not simultaneously represent 0, each of B₁ and B₂ independently represents a single bond or —(CH₂)ₖ—; wherein “k” represents an integer in a range of 0 to about 6, at least one —(CH₂)— in each of B₁ and B₂ is replaceable with —O—, —COO—, —OCO— or —CO—, D independently represents methacrylate or acrylate, and at least one hydrogen atom of each of A₁, A₂ and 2,5-thiophene is replaceable with F, Cl, OCH₃ or an alkyl group having 1 to about 6 carbon atoms.
